(19) United States
(12) Patent Application Publication
(10) Pub. No.: US 2006/0002941 A1
(43) Pub. Date: Jan. 5, 2006

(54) Compositions comprising immune response altering agents and methods of use

(75) Inventor: Gregory G. Mahairas, Seattle, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: VieVax Corp., Seattle, WA (US)

(21) Appl. No.: 11/041,893

(22) Filed: Jan. 24, 2005

Related U.S. Application Data
(60) Provisional application No. 60/538,713, filed on Jan. 23, 2004.
(60) Provisional application No. 60/616,855, filed on Oct. 6, 2004.

(51) Int. Cl. A61K 39/395; C07K 16/46

(52) U.S. Cl. 424/178.1; 530/391.1

(57) ABSTRACT

The present invention relates to immune response enhancing agents that alter an immune response generated against a heterologous target molecule. Compositions and methods of use of said immune response enhancing agents are also provided.

### FIELD OF THE INVENTION

The present invention relates generally to immune response altering agents that alter an immune response generated against a heterologous target molecule. In certain aspects, the immune response altering agent comprises multiple T cell epitopes and/or B cell epitopes, and/or Toll-like receptor (TLR) binding domains, that alter the immune response to a heterologous target antigen.

### DESCRIPTION OF THE RELATED ART

The extent of activation of either the humoral or cell-mediated branch of the immune system can determine the effectiveness of a vaccine against a particular disease. Furthermore, the development of immunologic memory by inducing memory-cell formation can be important for an effective vaccine against a particular disease (see for example, Paul, Fundamental Immunology, 4th Edition, 1999). The effectiveness of a vaccine at preventing or ameliorating the symptoms of a particular disease can depend on the type and strength of immune response generated by the vaccine.

Immune responses to many different antigens (e.g., antigens derived from infectious organisms, autoantigens or tumor antigens), while detectable, are frequently of insufficient magnitude or type to afford protection against a disease process mediated by agents (e.g., infectious microorganisms or tumor cells) expressing those antigens. In such situations, it is often desirable to administer to an appropriate subject, together with the antigen, an adjuvant that serves to enhance the immune response to the antigen in the subject.

### BRIEF SUMMARY OF THE INVENTION

One aspect of the present invention provides an immune response altering agent comprising a first domain comprising one or more T or B cell epitopes and/or Toll-like receptor (TLR) binding proteins or TLR binding domains thereof; and a second domain comprising a heterologous target molecule against which an immune response is desired; wherein the first domain alters an immune response in a subject against the heterologous target. In certain embodiments, the T cell or B cell, epitopes or TLR binding proteins are derived from more than one source. In another embodiment, the source is an infectious agent, including but not limited to a virus and/or a bacteria. In yet a further embodiment, the source is a tumor or a tumor antigen. In an additional embodiment, the source is an autoantigen. The source may also comprise a fungus, such as a yeast. In one embodiment, the source comprises mycoplasma. In a further embodiment, the source comprises a non-self antigen. In another embodiment the source comprises a self antigen.

In a further embodiment, the first domain of the immune response altering agent is covalently attached to the second domain via a peptide bond. In another embodiment, the first domain is chemically coupled to the second domain. In certain embodiments, the first domain is noncovalently attached to the second domain. In another embodiment, the first domain is mechanically attached to the second domain. The first domain may also be enzymatically attached to the second domain. In a further embodiment, the first domain is attached to the second domain via an electrostatic interaction or a hydrophobic interaction. In an additional embodiment, the first domain is attached to the second domain via biotin. In yet a further embodiment, the first domain is attached to the second domain via an antibody.

In certain embodiments, the heterologous target comprises a protein. In another embodiment, the heterologous target comprises a non-proteinaceous molecule. The heterologous target may also comprise a polysaccharide, a gycolipid, or a lipopolysaccharide. In another embodiment, the heterologous target comprises a tumor antigen. The heterologous target may also comprise an autoantigen. In a further embodiment, the heterologous target may comprise any one or more of a CMV protein, an RSV protein, a S. pneumoniae protein, a Chlamydia protein, a Hepatitis C protein, a Herpes virus protein, a Measles protein, or an influenza protein.

In one embodiment, the T cell or B cell epitopes or TLR binding domains of the immune response altering agent are generated synthetically. In another embodiment, the T cell or B cell epitopes or TLR binding domains are generated recombinantly. In certain embodiments, the T cell epitopes comprise CD4⁺ T helper cell epitopes. In a further embodiment, the T cell epitopes comprise CD8⁺ cytotoxic T cell epitopes. In another embodiment the T cell epitopes comprise both CD4⁺ T helper cell epitopes and CD8⁺ cytotoxic T cell epitopes.

In a further embodiment, the agent is a polynucleotide encoding a fusion protein. In one embodiment, the immune response altering agent is a fusion protein.

The present invention further provides compositions comprising immune response altering agents as described herein. In certain embodiments, the compositions comprising an immune response altering agent as described herein are in combination with a physiologically acceptable excipient. Further compositions of the present invention provide for an immune response altering agent in combination with an adjuvant.

In one aspect of the present invention, the immune response altering agent is attached to a targeting molecule for targeting the agent to a cell, tissue, or organ of interest. In this regard, the immune response altering agent may be attached to an antibody using methods known in the art and described herein. In this regard, the immune response altering agent can be targeted to a cell or tissue of interest using an antibody. In certain embodiments, the agent attached to antibody may induce antibody dependent cellular cytotoxicity (ADCC) or similar immune effects.

An additional aspect of the present invention provides a method for altering an immune response to a heterologous target comprising administering to a subject an immune response altering agent as described herein. In one embodiment, the target comprises an autoantigen, a tumor antigen, or an antigen derived from an infectious agent. In one embodiment, the immune response is altered from a Th2 type response to a Th1 type response.

A further aspect of the present invention provides a method for inducing an immune response to a target comprising administering to a subject an immune response altering agent as described herein. In one embodiment, the immune response comprises a CD8 cytotoxic T cell mediated response. In a further embodiment, the immune response comprises a CD4 T helper cell mediated response. In yet a further embodiment, the immune response comprises predominantly a Th1 type response or predominantly a Th2 type response.

An additional aspect of the present invention provides a T cell epitope cassette comprising multiple T cell epitopes, wherein said cassette alters an immune response to a heterologous target when administered as a fusion with, or attached to the heterologous target.

A further aspect of the present invention provides a composition comprising a heterologous target molecule and one or more first domains, said one or more first domains comprising a polypeptide sequence selected from the group consisting of either one of the full length polypeptide sequences set forth in SEQ ID NOs:214-215 (ESAT 6 and CFP10); and (ii) a fragment of either one of the full length polypeptide sequences set forth in SEQ ID NO:214-215, wherein the fragment induces an immune response that is not substantially reduced as compared to an immune response induced by the full length polypeptide. In one embodiment, the one or more first domains are not fused or otherwise attached to the heterologous target molecule. In one embodiment the polypeptide comprises the sequence set forth in SEQ ID NO:214. In an additional embodiment, the polypeptide comprises the sequence set forth in SEQ ID NO:215. In a further embodiment, the polypeptide comprises the sequence set forth in SEQ ID NO:214 and the sequence set forth in SEQ ID NO:215. In one embodiment, the fragment consists of at least 9 contiguous residues. In a further embodiment, the fragment consists of at least 20 contiguous residues. As discussed further herein, the fragments can be identified using any number of peptide mapping assays known in the art. In certain embodiments, the first domain comprises at least one polypeptide comprising any one or more of the sequences set forth in SEQ ID NOs:216-293.

One aspect of the present invention provides a method for inducing or enhancing an immune response to a heterologous target molecule in an individual comprising administering to the individual a composition comprising: the heterologous target molecule; and one or more first domains, said one or more first domains comprising a polypeptide sequence selected from the group consisting of: either one of the full length polypeptide sequences set forth in SEQ ID NOs:214-215; and a fragment of either one of the full length polypeptide sequences set forth in SEQ ID NO:214-215, wherein the fragment induces an immune response that is not substantially reduced as compared to an immune response induced by the full length polypeptide. In one embodiment, the one or more first domains are not fused or otherwise attached to the heterologous target molecule. In one embodiment, the polypeptide comprises the sequence set forth in SEQ ID NO:215. In a further embodiment, the polypeptide comprises the sequence set forth in SEQ ID NO:216. In certain embodiments, the polypeptide comprises the sequence set forth in SEQ ID NO:215 and the sequence set forth in SEQ ID NO:216. In yet a further embodiment, the fragment consists of at least 9 contiguous residues. In another embodiment, the fragment consists of at least 20 contiguous residues. In certain embodiments, the first domain comprises at least one polypeptide comprising any one or more of the sequences set forth in SEQ ID NOs:216-293. In a further embodiment, the immune response is a predominantly Th1-type response and in yet another embodiment, the immune response is a predominantly Th2-type response. In certain embodiment, the immune response is a predominantly Th0-type response. In one embodiment, the immune response is a CD4⁺ T cell response. In another embodiment, the immune response is a CD8⁺ T cell response. In yet an additional embodiment, the target molecule comprises an antigen selected from the group consisting of a viral coat protein, influenza neuraminidase, influenza hemmaglutinin, HIV gp160 or derivatives thereof, SARS coat protein, Herpes virion proteins, WNV capsid proteins, pneumococcal PsaA, PspA, LytA, Nisseria gonnorhea OMP or Nisseria gonnorhea surface proteases.

A further aspect of the present invention provides a method for inducing or enhancing an immune response to a target molecule in an individual comprising administering to the individual a composition comprising the target molecule; and one or more polypeptides or fragments thereof, wherein said one or more polypeptides or fragments thereof comprise at least one T cell epitope. In one embodiment, the target molecule is a non protein antigen. In this regard, the non protein antigen includes, but is not limited to, a bacterial polysaccharide, a glycolipid, a lipopolysaccharide, or a lipoprotein.

These and other embodiments of the present invention will become apparent upon reference to the following detailed description.

### BRIEF DESCRIPTION OF THE SEQUENCE IDENTIFIERS

SEQ ID NOs: 1-213 are illustrative peptide epitope sequences as set forth in Table 1.

SEQ ID NO:214 is the amino acid sequence of Mycobacteria tuberculosis Early Secretory Antigenic Target 6 (ESAT-6).

SEQ ID NO:215 is the amino acid sequence of Mycobacteria tuberculosis culture filtrate protein 10 (CFP 10).

SEQ ID NOs:216-236 are overlapping peptides spanning the entire Mycobacteria tuberculosis ESAT-6 protein. Each peptide consists of 15 amino acids and the peptides overlap by 11 amino acids.

SEQ ID NOs:237-244 are overlapping peptides spanning the entire Mycobacteria tuberculosis ESAT-6 protein. Each peptide consists of 20 amino acids and the peptides overlap by 10 amino acids.

SEQ ID NOs:245-262 are overlapping peptides spanning the entire Mycobacteria tuberculosis ESAT-6 protein. Each peptide consists of 10 amino acids and the peptides overlap by 5 amino acids.

SEQ ID NOs:263-271 are overlapping peptides spanning the entire Mycobacteria tuberculosis CFP10 protein. Each peptide consists of 20 amino acids and the peptides overlap by 10 amino acids.

SEQ ID NOs:272-293 are overlapping peptides spanning the entire Mycobacteria tuberculosis CFP10 protein. Each peptide consists of 15 amino acids and the peptides overlap by 11 amino acids.

SEQ ID NO:294 is an amino acid sequence of a flagellin protein that is conserved in Salmonella typhimurium species 1 and 2, as well as other species of S. typhimurium.

SEQ ID NO:295 is an amino acid sequence of the S. typhimurium flagellar filament protein (fliC) gene, a major component of bacterial flagellin.

### DETAILED DESCRIPTION OF THE INVENTION

The present invention relates generally to immune response altering agents. These agents can be used to alter an immune response to a target molecule of interest. As such, immune response altering agents are useful in the context of any number of disease settings as discussed further herein.

Generally, immune response altering agents are comprised of a first domain and a second domain wherein the first domain comprises at least one T cell epitope and/or B cell epitope, and/or Toll-like receptor binding proteins, or binding domains thereof, derived from any number of sources as described herein. In certain embodiments, the first domain comprises a protein that contains at least one T or B cell epitope, such as Mycobacteria tuberculosis Early Secretory Antigenic Target 6 (ESAT-6) or culture filtrate protein 10 (CFP10). In further embodiments, the first domain comprises Toll-like receptor binding proteins, such as bacterial flagellin proteins, or Toll-like receptor binding domains thereof. Illustrative TLR-like binding domains include the amino acid sequences set forth in SEQ ID NOs:294 and 295. The second domain comprises a heterologous target molecule against which an immune response is to be generated, enhanced, or otherwise altered (e.g., downregulated if an aberrant immune response against the target is present). The target molecule of the present invention is heterologous with respect to the epitopes and/or TLR binding proteins or binding domains thereof, comprised in the first domain.

Generally, the terms used herein are terms of art and should be construed as such unless otherwise noted. The following brief definitions are provided for convenience.

The terms “identical” or percent “identity”, in the context of two or more peptide sequences, refer to two or more sequences or subsequences that are the same or have a specified percentage of amino acid residues that are the same, when compared and aligned for maximum correspondence over a comparison window, as measured using any one of numerous sequence comparison algorithms known to the skilled person using default program parameters or by manual alignment and visual inspection.

The phrases “isolated” or “biologically pure” refer to material which is substantially or essentially free from components which normally accompany the material as it is found in its native state. Thus, isolated peptides in accordance with the invention preferably do not contain materials normally associated with the peptides in their in situ environment.

“Major histocompatibility complex” or “MHC” is a cluster of genes that plays a role in control of the cellular interactions responsible for physiologic immune responses. In humans, the MHC complex is also known as the HLA complex. For a more detailed description of the MHC and HLA complexes, see for example, Paul, Fundamental Immunology (4th ed. 1999).

“Human leukocyte antigen” or “HLA” is a human class I or class II major histocompatibility complex (MHC) protein (see, e.g., Stites, et al., Immunology, (8th ed., 1994).

The term “motif” is known to the skilled artisan and generally refers to the pattern of residues in a peptide of defined length, usually a peptide of from about 8 to about 13 amino acids for a class I HLA motif and from about 6 to about 25 amino acids for a class II HLA motif, which is recognized by a particular HLA molecule. Peptide motifs are typically different for each protein encoded by each human HLA allele and differ in the pattern of the primary and secondary anchor residues.

A “supermotif” is a peptide binding specificity shared by HLA molecules encoded by two or more HLA alleles. Thus, a preferably is recognized with high or intermediate affinity (as defined herein) by two or more HLA antigens.

“Cross-reactive binding” indicates that a peptide is bound by more than one HLA molecule; a synonym is degenerate binding.

“Immune response” as used herein, refers to activation of cells of the immune system, including but not limited to, T cells, B cells, macrophages, and dendritic cells, such that a particular effector function(s) of a particular cell is induced. Effector functions may include, but are not limited to, presentation of antigen, proliferation, secretion of cytokines, secretion of antibodies, expression of regulatory and/or adhesion molecules, expression of activation molecules, and the ability to induce cytolysis.

“Protective immune response” refers to a cytotoxic T lymphocyte (CTL) and/or a helper T lymphocyte (HTL) response to an antigen derived from an infectious agent or a tumor antigen, which prevents or at least partially arrests disease symptoms or progression. The immune response may also include an antibody response which has been facilitated by the stimulation of helper T cells.

“Synthetic peptide” refers to a peptide that is not naturally occurring, but is man-made using such methods as chemical synthesis or recombinant DNA technology.

As used herein, the term “expression vector” is intended to refer to a nucleic acid molecule capable of expressing a protein of interest, such as a MHC class I or class II epitope or cassette of multiple epitopes, in an appropriate target cell. An expression vector can be, for example, a plasmid or virus, including DNA or RNA viruses. The expression vector contains such a promoter element to express an antigen of interest in the appropriate cell or tissue in order to stimulate a desired immune response.

### The First Domain

The first domain of the immune response altering agents of the present invention comprises one or more T cell epitopes, and/or one or more B cell epitopes, and/or one or more TLR-binding domains. Illustrative epitopes include, but are not limited to, those described in Table 1 and set forth in SEQ ID NOs: 1-213. With regard to a particular amino acid sequence, an “epitope” is a set of amino acid residues which is involved in recognition by a particular immunoglobulin, or in the context of T cells, those residues necessary for recognition by T cell receptor proteins and/or Major Histocompatibility Complex (MHC) receptors. In an immune system setting, in vivo or in vitro, an epitope is the collective features of a molecule, such as primary, secondary and tertiary peptide structure, and charge, that together form a site recognized by an immunoglobulin, T cell receptor or HLA molecule. Throughout this disclosure epitope and peptide are often used interchangeably. It is to be appreciated, however, that isolated or purified protein or peptide molecules larger than and comprising an epitope of the invention are still within the bounds of the invention. For example, full length isolated proteins that contain at least one T cell or B cell epitope, such as ESAT6, CFP1O, heat shock proteins (HSPs), outer membrane proteins (Omps) and other proteins of the like, that are capable of inducing a desired immune response are also contemplated for use in the context of the present invention.

TABLE 1
Illustrative Epitopes

| | |
|---|---|
| | SEQ |
| | ID |
| Epitope Amino Acid Sequence | NO: |

| | |
|---|---|
| BACTERIA | |
| Mycobacterium leprae | |
| HSP65 T cell epitopes | |
| LEDPYEKIGAELVKEV | 1 |
| EQIAATAAISAGDQS | 2 |
| AGDQSIGDLIAEAMD | 3 |
| VEGAGDTDAIAGRVA | 4 |
| AGGVAVIKAGAATEV | 5 |
| GDEATGANIVKVALE | 6 |
| LQNAASIAGLFLTTE | 7 |
| AGGGVTLLQAAPALD | 8 |
| RVAQIRTEIENSD | 9 |
| LLQAAPALDKLKL | 10 |
| PEKTAAPASDPTG | 11 |
| Mycobacterium tuberculosis | |
| HSP65 T cell epitopes | |
| LEDPYEKIGAELVKEV | 12 |
| EQIAATAAISAGDQS | 13 |
| AGDQSIGDLIAEAMD | 14 |
| VEGAGDTDAIAGRVA | 15 |
| AGGVAVIKAGAATEV | 16 |
| GDEATGANTVKVALE | 17 |
| LQNAASIAGLFLTTE | 18 |
| AGGGVTLLQAAPALD | 19 |
| RVAQIRTEIENSD | 20 |
| LLQAAPALDKLKL | 21 |
| PEKTAAPASDPTG | 22 |
| Mycobacterium tuberculosis | |
| Ag85A T cell epitope | |
| LPAKFLEGF | 23 |
| Mycobacterium tuberculosis | |
| Ag85B/MPT59 T cell epitopes | |
| YLQVPSPSMGRDIKVQFQ | 24 |
| GRDIKVQFQSGGNNSPAV | 25 |
| GCQTYKWETLLTSELPQW | 26 |
| IPAEFLENF | 27 |
| Mycobacterium tuberculosis | |
| Ag85C T cell epitopes | |
| WPTLIGLAM | 28 |
| IPAKFLEGL | 29 |
| Mycobacterium tuberculosis | |
| Ag85ABC T cell epitopes | |
| MPVGGQSSF | 30 |
| MPVGGQSSFY | 31 |
| Mycobacterium tuberculosis | |
| Rv3019c T cell epitope | |
| MSQIMYNYPAMMAHAGDM | 32 |
| ITYQGWQTQWNQALED | 33 |
| Mycobacterium tuberculosis | |
| 16 kDa protein antigen CD8 T cell epitopes | |
| ATFAAPVALAA | 34 |
| SGATIPQGEQS | 35 |
| Mycobacterium tuberculosis/M. bovis | |
| MPB70 T cell epitopes | |
| AVAASNNPELTTLTAALSGQLNPQV | 36 |
| ALSGQLNPQVNLVDTLNSGQY | 37 |
| FSKLPASTIDELKTNSSLLTSILTYH | 38 |
| GNADVVCGGVSTANATVYMIDSVL | 39 |
| ATTVYMIDSVLMPPA | 40 |
| Mycobacterium tuberculosis/M. bovis | |
| ESAT6 T cell epitopes | |
| MTEQQWNFAGIEAAASAIQG | 41 |
| EQQWNFAGIEAAA | 42 |
| WNFAGIEAA | 43 |
| VQGVQQKWDATATELNNALQ | 44 |
| AWGGSGSEAYQGVQQKWDATATEL | 45 |
| QGVQQKWDATATELNNALQNLART | 46 |
| LARTISEAGQAMASTEGNVTGMFA | 47 |
| ESAT-6 B cell epitope | |
| EQQWNFAGIEAAA | 48 |
| Streptococcus mutans | |
| SAI/II protein antigen T cell epitopes 816-1213 | |
| NNNDVNIDRTLVAKQSVVKF | 49 |
| QLKTADLPAGRDETTSFVLV | 50 |
| LATFNADLTKSVATIYPTVV | 51 |
| Chlamydia pneumoniae | |
| CD8 T cell epitopes | |
| GDYVFDRI | 52 |
| SLLGNATAL | 53 |
| QAVANGGAI | 54 |
| RGAFCDKEF | 55 |
| CYGRLYSVKV | 56 |
| KYNEEARKKI | 57 |
| GPKGRHVVI | 58 |
| Corynebacterium diptheriae | |
| diptheria toxin T cell epitope | |
| NLFQVVHWSYNRPAYSPGYV | 59 |
| Esherichia coli | |
| OmpF T cell epitopes | |
| AQTGNKTRLAFAGLKYADVG | 60 |
| FDFGLRPSTAYTKSKAKDVE | 61 |
| FEVGATYYFNKNMSTYVDYII | 62 |
| NKNMSTYVDYIINQIDSDNK | 63 |
| Eshericia coli | |
| beta galactosidase T cell epitope | |
| TPHPARIGL | 64 |
| Salmonella typhimurium | |
| SipC CD4 T cell epitope | |
| LIQCMLKKTMLSINQ | 65 |
| Listeria monocytogenes | |
| Listeriolysin O T cell epitope | |
| GYKDGNEYI | 66 |
| Borrelia burgdorferi | |
| OspA T cell epitope | |
| VVKEGTVTLSKNISKSGEVS | 67 |
| VIRUSES | |
| Lymphocytic choriomeningitis | |
| virus nucleoprotein T cell epitopes | |
| FQPQNGQFI | 68 |
| RPQASGVYM | 69 |
| Lymphocytic choriomeningitis | |
| virus nucleoprotein T cell epitopes | |
| PYIACRTSI | 70 |
| MPYIACRTSI | 71 |
| WPYIACRTSI | 72 |
| Lassa Fever Virus Nucleoprotein T cell epitopes | |
| FGTMPSLTLACLT | 73 |
| FGTMPSLTIACMC | 74 |
| QGQVDLNDAVQAL | 75 |
| QGQADLNDVIQSL | 76 |
| ALGMFISDTPGER | 77 |
| SLGMFVSDTPGER | 78 |
| QLDPNAKTWMDIE | 79 |
| NLIPNAKTWMDIE | 80 |
| VWDQYKDLCHMHT | 81 |
| VWDQFKDLCHMHT | 82 |
| IWDEYKHLCRMHT | 83 |
| HIV-1 Nef Peptide T cell epitopes | |
| FPVTPQVP | 84 |
| FPVTPRVPL | 85 |
| TPQVPLRPM | 86 |
| AVDLSHFLK | 87 |
| YPLTFGWCY | 88 |
| PLTFGWCYK | 89 |
| LTFGWCYKL | 90 |
| HIV-1 Gag peptide T cell epitopes | |
| GEIYKRWII | 91 |
| EIYKRWIIL | 92 |
| KRWIILGLNK | 93 |
| ILGLNKIV | 94 |
| ILGLNKIVRMY | 95 |
| HIV-1 T cell epitopes | |
| Hepatitis B virus | |
| surface antigen T cell epitopes (T helper) | |
| QAGFFLLTRILTIPQSLD | 96 |
| SCCCTKPTDGNCTCIPIPSS | 97 |
| WEWASVRFSWLS | 98 |
| LPLLPIFFCLWVYI | 99 |
| Human Papillomavirus E7 protein T cell epitope | |
| RAHYNIVTF | 100 |
| GQAEPDRAHYNIVTFCCKCDSTLRLCVQSTHVDIR | 101 |
| Epstein Barr virus A3 protein T cell epitopes | |
| RRIYDLIEL | 102 |
| RKIYDLIEL | 103 |
| FRKAQIQGL | 104 |
| HRCQAIRK | 105 |
| RRARSLSAERY | 106 |
| Epstein Barr virus | |
| Latent membrane protein T cell epitope | |
| RRRWRRLTV | 107 |
| Epstein Barr virus | |
| Latent membrane protein 1 T cell epitopes | |
| DWTGGALLVLYSFALML | 108 |
| ALLVLYSFAL | 109 |
| LLVLYSFAL | 110 |
| ALLVLYSFA | 111 |
| VLYSFALML | 112 |
| LVLGIWIYLLEMLWRRLG | 113 |
| YLLEMLWRL | 114 |
| LIIALYLQQNWWTLLVD | 115 |
| IALYLQQNW | 116 |
| ALYLQQNWW | 117 |
| YLQQNWWTL | 118 |
| QNWWTLLVD | 119 |
| LYLQQNWWT | 120 |
| LIWMYYHGQRHSDEHHH | 121 |
| QRHSDEHHH | 122 |
| GQRHSDEHH | 123 |
| YYHGQRHSD | 124 |
| WMYYHGQRH | 125 |
| TDDSGHESDSNSNEGRH | 126 |
| ESDSNSNEG | 127 |
| DSNSNEGRH | 128 |
| PHSPSDSAGNDGGPPQL | 129 |
| AGNDGGPPQ | 130 |
| PSDSAGNDG | 131 |
| RHSDEHHHDDSLPHPQQ | 132 |
| Epstein Barr virus | |
| Nuclear antigen 6 T cell epitope | |
| EENLLDVFRM | 133 |
| Epstein Barr virus immediate-early | |
| transactivator protein (Rta) T cell epitope | |
| LVSDYCNVLNKEFTA | 134 |
| FFIQAPSNRVMIPAT | 135 |
| RVMIPATIGTAMYKL | 136 |
| KHSRVRAYTYSKVLG | 137 |
| RALIKTLPRASYSSH | 138 |
| ERPIFPHPSKPTFLP | 139 |
| EVCQPKRIRPFHPPG | 140 |
| QKEEAAICGQMDLSH | 141 |
| DYCNVLNKEF | 142 |
| ATIGTAMYK | 143 |
| Epstein Barr virus | |
| Immunodominant latent-cycle epitopes | |
| QAKWRLQTL | 144 |
| FLRGRAYGL | 145 |
| IVTDFSVIK | 146 |
| AVFDRKSDAK | 147 |
| RRIYDLIEL | 148 |
| RRARSLSAERY | 149 |
| Epstein Barr virus | |
| Subdominant latent-cycle epitopes | |
| LLWTLVVLL | 150 |
| CLGGLLTMV | 151 |
| IEDPPFNSL | 152 |
| SSCSSCPLSKI | 153 |
| TYGPVFMCL | 154 |
| Epstein Barr virus Lytic-cycle epitopes | |
| APENAYQAY | 155 |
| RAKFKQLL | 156 |
| GLCTLVAML | 157 |
| TLDYKPLSV | 158 |
| Epstein Barr virus | |
| QNGALAINTE | 159 |
| LLDFVRFMGV | 160 |
| EENLLDFVRF | 161 |
| Epstein Barr virus | |
| tegument protein T cell epitope | |
| HPLTNNLPL | 162 |
| Hantaan Virus Nucleocapsid protein | |
| NAHEGQLVI | 163 |
| ISNQEPLKL | 164 |
| Hepatitis C Virus nucleoprotein | |
| GYKVLVLNPSVAAT | 165 |
| Dengue Virus capsid protein | |
| LIGFRKEIGRMLNIL | 166 |
| KGPLRMVLAFITFLR | 167 |
| Rotavirus VP6 CD4+ T cell epitopes | |
| RNFDTIRLSFQLVER | 168 |
| RLSFQLVRPPNMTP | 169 |
| VRPPNMTPAVANLF | 170 |
| Measles Virus T cell epitope | |
| LSEIKGVIVHRLEGV | 171 |
| B cell epitope | |
| INQDPDKILTY | 172 |
| Canine Distemper Virus T cell epitope | |
| LSEVKGVIVHRLEAV | 173 |
| B cell epitope | |
| INQSPDKILTY | 174 |
| PARASITES | |
| Trypanosoma cruzi | |
| trans-sialidase (TS) gene T cell epitope | |
| IYNVGQVSI (CD8) | 175 |
| Trypanosoma cruzi | |
| surface glycoprotein T cell epitope | |
| SHNFTLVASVIIEEA | 176 |
| LVASVIIEEAPSGNT | 177 |
| Toxoplasma gondii | |
| ROP2 protein antigen T cell epitopes | |
| TDPGDVVIEELFNRIPETSV | 178 |
| LQLIRLAASLQHYGLVHA | 179 |
| IEWIYRRCKNIPQPVRALLEGFLR | 180 |
| Babesia bovis RAP1 T cell epitopes (CD4+) | |
| EYLVNKVLYMATMNYKT | 181 |
| EAPWYKRWIKKFR | 182 |
| FREAPQATKHFL | 183 |
| FREAPQATKHFLDEN | 184 |
| FREAPQATKHFLGEN | 185 |
| FVVSLLKKNVVRDPESNDVENFASQYFYM | 186 |
| VNSEKVDADDAGNAETQQLPDAENEVRADD | 187 |
| Plasmodium vivax MSP-1 T cell eptitopes | |
| NFVGKFLELQIPGHTDLLHL | 188 |
| FNQLMHVINFHYDLLRANVH | 189 |
| LDMLKKVVLGLWKPLDNIKD | 190 |
| LEYYLREKAKMAGTLIIPES | 191 |
| KKIKAFLETSNNKAAAPAQS | 192 |
| SKDQIKKLTSLKNKLERRQN | 193 |
| Anaplasma marginale MSP1A T cell epitopes | |
| Plasmodium falciparum MSP-1 T cell epitopes | |
| DPNANPNVDPNANPNV | 194 |
| Plasmodium falciparum MSP-1 T cell epitopes | |
| FGYRKPLDNIKDNVGKMEDYIKK | 195 |
| SKLNSLNNPHNVLQNFSVFFNKK | 196 |
| Plasmodium falciparum MSP-1 T cell epitopes | |
| GYRKPLDNIKDNVGKMEDYIKK | 197 |
| KLNSLNNPHNVLQNFSVFFNK | 198 |
| TKILLKHYKGLVKYYNGESSP | 199 |
| HGFKYLIDGYEEINELLYKLN | 200 |
| Plasmodium falciparum | |
| MSP-1 T cell/B cell epitopes | |
| VTHESYQELVKKLEALEDAV | 201 |
| GLFHKEKMILNEEEITTKGA | 202 |
| Plasmodium falciparum ABRA T cell epitope | |
| DSNIMNSINNVMDEIDFFEK | 203 |
| Plasmodium falciparum SERA T cell epitope | |
| DDYTEYKLTESIDNILVKMFKTN | 204 |
| Plasmodium falciparum | |
| Liver stage 1 antigen T cell epitopes | |
| LTMSNVKNVSQTNFKSLLRNL | 205 |
| HTLETVNISDVNDFQISKY | 206 |
| DDEDLDEFKPIVQYDNFQD | 207 |
| EENIGIKELEDLIEKNENL | 208 |
| DDLDEGIEKSSEELSEEK | 209 |
| IKKGKKYEKTKDNNF | 210 |
| DNEILQIVDELSEDITKYFMKL | 211 |
| EQQQSDLEQERLAKEKLQEQQSDLEQERRAKEKLQ | 212 |
| OTHER | |
| Chicken Ovalbumin T cell epitope | |
| SIINFEKL | 213 |

In another embodiment, full length Toll-like receptor binding proteins or TLR-binding domains thereof, such as isolated flagellin proteins or TLR-binding portions thereof, are contemplated for use in the context of the present invention. As such, the first domain may comprise one or more TLR-binding proteins or TLR-binding domains thereof. As would be recognized by the skilled artisan, any protein that recognizes any of the Toll-like receptors, is contemplated herein. Exemplary TLR-binding proteins include bacterial flagellin proteins, or portions thereof that bind TLRs, such as those set forth in SEQ ID NO:294 and 295 (see e.g., K. D. Smith et al., 2003 Nature Immunology, 4(12):1247-1253). Toll-like receptor binding proteins, or TLR-binding domains thereof, include proteins or domains/motifs thereof, that bind to or other recognize Toll-like receptors 1, 2, 3, 4, 5, 6, 7, 8, 9, 10, and 11 (see also Akira, S. (2003). Curr Opin Immunol 15(1): 5-11; Kaisho, T. and S. Akira (2003). Curr Mol Med 3(4): 373-85.). TLR-binding activity can be measured using a variety of assays known in the art, for example, by measuring IFN-γ production by macrophages or other cells activated by TLR-binding proteins. Other assays to test TLR-binding activity are described, for example, in K. D. Smith, et al., 2003, supra.

Proteins containing at least one epitope for use in the present invention can be identified using a variety of techniques known in the art. Illustrative methods are described in Current Protocols in Immunology, Edited by: John E. Coligan, Ada M. Kruisbeek, David H. Margulies, Ethan M. Shevach, Warren Strober (2001 John Wiley & Sons, NY, N.Y.) Ausubel et al. (2001 Current Protocols in Molecular Biology, Greene Publ. Assoc. Inc. & John Wiley & Sons, Inc., NY, N.Y.); Sambrook et al. (1989 Molecular Cloning, Second Ed., Cold Spring Harbor Laboratory, Plainview, N.Y.); Maniatis et al. (1982 Molecular Cloning, Cold Spring Harbor Laboratory, Plainview, N.Y.) and elsewhere. Illustrative methods useful in this context include intracellular cytokine staining (ICS), ELISPOT, proliferation assays, cytotoxic T cell assays including chromium release or equivalent assays, and gene expression analysis using any number of polymerase chain reaction (PCR) or RT-PCR based assays.

Epitopes of the present invention may be identified using any number of techniques known in the art, such as those described by: Lamb J R, et al. 1989. Rev. Infect. Dis. March-April: Suppl 2:s443-447; Lamb J R, et al. 1987. EMBO J. May; 6(5):1245-1249; Lamb J R, et al. 1986. Lepr. Rev. Dec.; Suppl 2:131-137; Mehra V, et al. 1986. Proc. Natl. Acad. Sci. Sep.; 83(18): 7013-7; Horsfall A C, et al. 1991. Immunol. Today. July; 12(7):211-3; Rothbard J B and Lamb J R. 1990. Curr Top Microbiol Immunol 155:143-52; Singh H and Raghava G P. 2001. Bioinformatics 17:1236-1237; DeGroot A S, et al. Vaccine 19:4385-4395; DeLalla C, et al. 1999. J. Immunol. 163:1725-1729; Cochlovius B, et al. 2000. J. Immunol. 165:4731-4741; Consogno G, et al. 2003. Blood 101:1039-1044; Roberts C G, et al. 1996. AIDS Res. Hum. Retrovir. 12:593-610; Kwok W, et al. 2001. Trends Immunol. 22:583-588; Novak E J, et al. 2001. J. Immunol. 166:6665-6670.

An epitope of the present invention may comprise a naturally occurring or naturally processed epitope as defined using any number of assays known to the skilled artisan and as described herein. Assays for identifying epitopes are known to the skilled artisan and are described, for example, in Current Protocols in Imnmunology, John E. Coligan, Ada M. Kruisbeek, David H. Margulies, Ethan M. Shevach, and Warren Strober (Eds), John Wiley & Sons, New York. N.Y. Epitopes may be identified using intracellular cytokine staining and flow cytometric analysis such as described in Hoffineister B., et al., Methods. 2003 March; 29(3):270-281; Maecker H T, et al. J Immunol Methods. 2001 September 1;255(1-2):27-40. Similarly, proteins, peptides, overlapping peptides, or pools of peptides can be used in standard chromium release and/or proliferation assays to identify epitopes.

In those cases where antigen-specific T cell lines or clones are available, for example tumor-infiltrating lymphocytes (TIL) or virus-specific CTL, these cells can be used to screen for the presence of the relevant epitopes using target cells prepared with specific antigens. Such targets can be prepared using random, or selected synthetic peptide libraries, which would be utilized to sensitize the target cells for lysis by the CTL. Another approach to identify the relevant epitope when T cell lines or clones are available is to use recombinant DNA methodologies. Gene, or preferably cDNA, libraries from CTL-susceptible targets are first prepared and transfected into non-susceptible target cells. This allows the identification and cloning of the gene coding the protein precursor to the peptide containing the CTL epitope. The second step in this process is to prepare truncated genes from the relevant cloned gene, in order to narrow down the region that encodes for the CTL epitope. This step is optional if the gene is not too large. The third step is to prepare synthetic peptides of approximately 10-20 amino acids of length, overlapping by 5-10 residues, which are used to sensitize targets for the CTL. When a peptide, or peptides, is shown to contain the relevant epitope, smaller peptides can be prepared to establish the peptide of minimal size that contains the epitope. These epitopes are usually contained within 9-10 residues for CTL epitopes and up to 20 or 30 residues for HTL epitopes.

Alternatively, epitopes may be defined by direct elution of peptides bound by particular MHC molecule and direct sequencing of the peptides (see, for example, Engelhard V H, et al., Cancer J. 2000 May; 6 Suppl 3:S272-80). Briefly, the eluted peptides are separated using a purification method such as HPLC, and individual fractions are tested for their capacity to sensitize targets for CTL lysis or to induce proliferation of cytokine secretion in HTL. When a fraction has been identified as containing the peptide, it is further purified and submitted to sequence analysis. The peptide sequence can also be determined using tandem mass spectrometry. A synthetic peptide is then prepared and tested with the CTL or HTL to corroborate that the correct sequence and peptide have been identified.

Epitopes may be identified using computer analysis, such as the Tsites program (see Rothbard and Taylor, EMBO J. 7:93-100, 1988; Deavin et al., Mol. Immunol. 33:145-155, 1996), which searches for peptide motifs that have the potential to elicit Th responses. CTL peptides with motifs appropriate for binding to murine and human class I or class II MHC may be identified according to BIMAS (Parker et al., J. Immunol. 152:163, 1994) and other HLA peptide binding prediction analyses. Briefly, the protein sequences for example from viral or tumor cell components are examined for the presence of MHC-binding motifs. These binding motifs which exist for each MHC allele are conserved amino acid residues, usually at positions 2 (or 3) and 9 (or 10) for MHC class I binding peptides of 9-10 residues long. Synthetic peptides are then prepared of those sequences bearing the MHC binding motifs, and subsequently are tested for their ability to bind to MHC molecules. The MHC binding assay can be carried out either using cells which express high number of empty MHC molecules (cellular binding assay), or using purified MHC molecules. Lastly, the MHC binding peptides are then tested for their capacity to induce a CTL response in naive individuals, either in vitro using human lymphocytes, or in vivo using HLA-transgenic animals. These CTL are tested using peptide-sensitized target cells, and targets that naturally process the antigen, such as viral infected cells or tumor cells. To further confirm immunogenicity, a peptide may be tested using an HLA A2 transgenic mouse model and/or any of a variety of in vitro stimulation assays.

Epitopes of the present invention may also be identified using a peptide motif searching program based on algorithms developed by Rammensee, et al. (Hans-Georg Rammensee, Jutta Bachmann, Niels Nikolaus Emmerich, Oskar Alexander Bachor, Stefan Stevanovic: SYFPEITHI: database for MHC ligands and peptide motifs. Immunogenetics (1999) 50: 213-219); by Parker, et. al. (Supra), or using methods such as those described by Doytchinova and Flower in Immunol Cell Biol. 2002 June; 80(3):270-9 and Blythe M J, Doytchinova I A, Flower D R. JenPep: a database of quantitative functional peptide data for immunology. Bioinformatics (2002), 18, 434-439.

In certain embodiments, an epitope may comprise a variant of a native epitope. A “variant,” as used herein, is a polypeptide (or a nucleic acid encoding such a polypeptide) that differs from a native polypeptide in one or more substitutions, deletions, additions and/or insertions, such that the immunogenicity of the polypeptide is retained (i.e., the ability of the variant to react with antigen-specific antisera and/or T-cell lines or clones is not substantially diminished relative to the native polypeptide). In other words, the ability of a variant to react with antigen-specific antisera and/or T-cell lines or clones may be enhanced or unchanged, relative to the native polypeptide, or may be diminished by less than 50%, and preferably less than 20% relative to the native polypeptide. In one embodiment the ability of a variant to react with antigen-specific antisera and/or T-cell lines or clones may be diminished by less than 30%, 25%, 20%, 19%, 18%, 17%, 16%, 15%, 10%, 9%, 8%, 7%, 6%, 5%, 4%, 3%, 2%, 1%, or 0.5 %, relative to the native polypeptide. In one embodiment the ability of a variant to react with antigen-specific antisera and/or T-cell lines or clones may be enhanced by at least 30%, 25%, 20%, 19%, 18%, 17%, 16%, 15%, 10%, 9%, 8%, 7%, 6%, 5%, 4%, 3%, 2%, 1%, or 0.5 %, relative to the native polypeptide. Such variants may generally be identified by modifying one of the above polypeptide sequences and evaluating the reactivity of the modified polypeptide with antisera and/or T-cells as described herein. In one embodiment of the present invention, a variant may be identified by evaluating its ability to bind to a human, murine, or nonhuman primate MHC molecule. In one preferred embodiment, a variant polypeptide has a modification such that the ability of the variant polypeptide to bind to a class I or class II MHC molecule is increased relative to that of a wild type (unmodified) polypeptide. The skilled artisan would recognize that any number of class I or class II MHC molecules can be used in the context of the invention, for example any HLA molecule as identified and available from the IMGT/HLA database (http://www.ebi.ac.uk/imgt/hla).

In a further embodiment, the ability of the variant polypeptide to bind to an HLA molecule is increased by at least 2 fold, preferably at least 3 fold, 4 fold, or 5 fold relative to that of a native polypeptide. It has been found, within the context of the present invention, that a relatively small number of substitutions (e.g., 1 to 3) within an epitope may serve to enhance the ability of the epitope to elicit an immune response. Suitable substitutions may generally be identified by using computer programs, as described above, and the effect confirmed based on the reactivity of the modified polypeptide with antisera and/or T-cells as described herein. Accordingly, within certain preferred embodiments, a variant in Which 1 to 3 amino acid resides within an epitope are substituted such that the ability to react with antigen-specific antisera and/or T-cell lines or clones is statistically greater than that for the unmodified polypeptide. Such substitutions are preferably located within an MHC binding site of the polypeptide, which may be identified as described above. Preferred substitutions allow increased binding to MHC class I or class II molecules.

In further embodiments, the present invention provides variants of TLR-binding proteins, or TLR-binding domains thereof. In this regard, a variant of a TLR-binding protein, or TLR-binding domain thereof, is a polypeptide that differs from a native polypeptide in one or more substitutions, deletions, additions and/or insertions, such that the TLR-binding activity of the polypeptide is retained. In other words, the ability of a variant to bind to its cognate TLR may be enhanced or unchanged, relative to the native polypeptide, or may be diminished by less than 50%, and preferably less than 20% relative to the native polypeptide. In one embodiment the ability of a variant to bind its cognate TLR may be diminished by less than 30%, 25%, 20%, 19%, 18%, 17%, 16%, 15%, 10%, 9%, 8%, 7%, 6%, 5%, 4%, 3%, 2%, 1%, or 0.5 %, relative to the native polypeptide. In one embodiment the ability of a variant to bind its cognate TLR may be enhanced by at least 30%, 25%, 20%, 19%, 18%, 17%, 16%, 15%, 10%, 9%, 8%, 7%, 6%, 5%, 4%, 3%, 2%, 1%, or 0.5 %, relative to the native polypeptide. Such variants may generally be identified by modifying a TLR-binding polypeptide or TLR-binding domain thereof as described herein, and evaluating the ability of such modified polypeptides to bind to cognate TLRs (see for example, K. D. Smith, et al, 2004, supra). In certain embodiments, the variant TLR polypeptide activity can be tested by measuring cytokine production by cells expressing appropriate TLRs (e.g., IFN-γ production by macrophages). Thus, in this regard, the ability of a variant to induce production of cytokines in an appropriate cell may be diminished by less than 30%, 25%, 20%, 19%, 18%, 17%, 16%, 15%, 10%, 9%, 8%, 7%, 6%, 5%, 4%, 3%, 2%, 1%, or 0.5 %, relative to the native polypeptide. In a further embodiment the ability of a variant to induce cytokine production in an appropriate cell may be enhanced by at least 30%, 25%, 20%, 19%, 18%, 17%, 16%, 15%, 10%, 9%, 8%, 7%, 6%, 5%, 4%, 3%, 2%, 1%, or 0.5 %, relative to the native polypeptide.

Certain variants contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser; tyr, thr; (3) val, ile, leu, met, ala, phe; (4) lys, arg, his; and (5) phe, tyr, trp, his. A variant may also, or alternatively, contain nonconservative changes. Variants may also (or alternatively) be modified by, for example, the deletion or addition of amino acids that have minimal influence on the immunogenicity, secondary structure and hydropathic nature of the polypeptide.

Epitopes of the present invention include but are not limited to epitopes derived from: infectious organisms such as any variety of viruses, such as, single stranded RNA viruses, single stranded DNA viruses, cytomegalovirus (CMV), Rous sarcoma virus (RSV), hepatitis A virus, hepatitis B virus (HBV), Hepatitis C (HCV), Herpes viruses, such as herpes simplex virus (HSV), Influenza viruses, west nile virus (WNV), Epstein-Barr virus (EBV), eastern equine encephalitis virus (EEEV), severe acute respiratory virus (SARS), human immunodeficiency virus (HIV), human papilloma virus (HPV), and human T cell lymphoma virus (HTLV); parasites (e.g., protozoan and metazoan pathogens such as Plasmodia species, Leishmania species, Schistosoma species, Trypanosoma species, flagellated protozoa Giardia duodenalis; Entamoebae), bacteria (e.g., eubacterial genera Acholeplasma, Anaeroplasma, Asteroleplasma, Mycoplasma, Spiroplasma and Ureaplasma; Mycobacteria, in particular, M. tuberculosis, Salmonella, Streptococci, E. coli, Staphylococci, Chlamydia species, Pseudomonads); fungi (e.g., Candida species, Aspergillus species and other yeast species), and Pneumocystis carinii.

In certain embodiments, the epitopes of the present invention are derived from the ESAT-6 protein, such as described in U.S. Pat. No. 6,537,552. In another embodiment, the epitopes may be derived from outer membrane proteins derived from bacterial pathogens.

Cytotoxic T lymphocytes (CTLs) and helper T lymphocytes (HTLs) are critical for immunity against infectious pathogens; such as viruses, bacteria, and protozoa; tumor cells; autoimmune diseases and the like. The present invention provides immune response altering agents that encode peptide epitopes which induce a CTL and/or HTL response to a heterologous target molecule. In certain aspects of the present invention, the immune response altering agents reduce a CTL and/or HTL response to a given heterologous target molecule.

In certain embodiments of the present invention, the T and/or B cell epitopes and/or TLR-binding proteins, or TLR-binding domains/portion thereof, may be combined or linked as a “cassette”. Cassettes of epitopes may comprise a combination of one or more HTL epitopes and/or one or more CTL epitopes and/or one or more TLR-binding domains. In certain embodiments, it may be desirable to include a universal HTL epitopes, or Pan DR epitopes (PADRE) such as those described in U.S. Pat. No. 5,736,142, or other “promiscuous” epitopes, such as those described in U.S. Pat. No. 6,419,931. About 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more different epitopes, either HTL and/or CTL, and/or TLR binding domains can be included in the cassette, along with other components, such as for example, an MHC targeting sequence, a lysosomal associated membrane protein-1 (LAMP-1) and the like. Additionally, in certain embodiments, TLR-binding proteins, such as flagellin proteins, or peptides derived therefrom, can be included in the first domain, for example as part of a cassette. For example, the flagellin peptides set forth in SEQ ID NOs:294 and 295 may be included in a cassette. In this regard, bacterial flagellin stimulate the toll-like receptor 5 (TLR5) thereby activating host inflammatory responses (see e.g., K. D. Smith, et al., 2003 Nature Immunology 4:1247-1253). Thus, the flagellin proteins can enhance the immune response altering and enhancing ability of the T cell epitopes as described herein. In certain embodiments, the flagellin proteins can be used in the first domain without T cell epitopes for a more general adjuvant-like effect. The skilled artisan would readily appreciate that the epitopes can have different HLA restriction. Additionally, epitopes of the present invention may either be derived from self or non-self antigens.

Thus, the first domain of the present invention may comprise isolated proteins or fragments or portions thereof and polynucleotides that encode such proteins. As used herein, the terms protein and polypeptide are used interchangeably. The terms “polypeptide” and “protein” encompass amino acid chains of any length, including full-length endogenous (i.e., native) proteins and variants of native polypeptides as described herein. The epitopes or proteins containing at least one epitope, and the TLR-binding proteins, or portions thereof, of the first domain, and any other desirable components such as targeting sequences, can be generated recombinantly as an expression vector as described further elsewhere herein (see “Polynucleotides encoding immune response altering agents and/or components thereof”.)

Linkages for epitopes, or proteins containing at least one epitope, in a cassette or for coupling to carriers or to heterologous target molecules can be provided in a variety of ways. For example, cysteine residues can be added at both the amino- and carboxy-termini, where the peptides are covalently bonded via controlled oxidation of the cysteine residues. Also useful are a large number of heterobifunctional agents which generate a disulfide link at one functional group end and a peptide link at the other, including N-succidimidyl-3-(2-pyridyldithio) proprionate (SPDP). This reagent creates a disulfide linkage between itself and a cysteine residue in one protein and an amide linkage through the amino on a lysine or other free amino group in the other. A variety of such disulfide/amide forming agents are known. See, for example, Immun. Rev. 62:185 (1982). Other bifunctional coupling agents form a thioether rather than a disulfide linkage. Many of these thioether forming agents are commercially available and include reactive esters of 6-maleimidocaproic acid, 2 bromoacetic acid, 2-iodoacetic acid, 4-(N-maleimido-methyl) cyclohexane-1-carboxylic acid and the like. The carboxyl groups can be activated by combining them with succinimide or 1-hydroxy-2-nitro-4-sulfonic acid, sodium salt. One coupling agent is succinimidyl 4-(N-maleimidomethyl) cyclohexane-l-carboxylate (SMCC). Of course, it will be understood that linkage should not substantially interfere with either of the linked groups to function as described, e.g., to function as an immune response altering agent.

A variety of cassettes encoding any number of different epitopes and other components (e.g., MHC targeting sequence, LAMP-1, TLR-binding proteins, such as flagellin proteins, or TLR-binding fragments thereof) can be tested for the ability to alter an immune responses using in vitro assays known to the skilled artisan, such as intracellular cytokine staining assays, chromium release assays, or T cell proliferation assays, cytokine assays, or macrophage IFN-γ production assays. A variety of immunological assays useful in the context of the present invention are described for example in Current Protocols in Immunology (John Wiley & Sons, Supra). Additionally, mouse models, including HLA transgenic mouse models, can be used to test for immunogenicity of the cassettes described herein.

In certain embodiments, an epitope or other protein or peptide of the present invention is synthetically designed, generated and/or modified. The epitopes or other protein or peptides described herein can be prepared “synthetically,” as described herein below, or by recombinant DNA technology. In certain embodiments, the polypeptides of the present invention of the first or second domain can be generated by combinatorial chemistry, such as described by R. A. Houghten, et al., Proc Natl Acad Sci USA. 1994 Nov 8;91(23):11138-42. Although the peptide will preferably be substantially free of other naturally occurring viral, bacterial, parasitic, tumor or self proteins and fragments thereof, in some embodiments the peptides can be synthetically conjugated to native fragments or particles. The term peptide is used interchangeably with polypeptide in the present specification to designate a series of amino acids connected one to the other by peptide bonds between the alpha-amino and alpha-carboxy groups of adjacent amino acids. The polypeptides or peptides can be a variety of lengths, either in their neutral (uncharged) forms or in forms which are salts, and either free of modifications such as glycosylation, side chain oxidation, or phosphorylation or containing these modifications, subject to the condition that the modification not destroy the biological activity of the polypeptides as herein described. The peptides of the invention can be prepared in a wide variety of ways. For example, the peptides can be synthesized in solution or on a solid support in accordance with conventional techniques. Various automatic synthesizers are commercially available and can be used in accordance with known protocols. See, for example, Stewart and Young, Solid Phase Peptide Synthesis, 2d. ed., Pierce Chemical Co. (1984); Tam et al., J. Am. Chem. Soc. 105:6442 (1983); Merrifield, Science 232:341-347 (1986); and Barany and Merrifield, The Peptides, Gross and Meienhofer, eds., Academic Press, New York, pp. 1-284 (1979).

The terms “homologous”, “substantially homologous”, and “substantial homology” as used herein denote a sequence of amino acids having at least 50% identity wherein one sequence is compared to a reference sequence of amino acids. The percentage of sequence identity or homology is calculated by comparing one to another when aligned to corresponding portions of the reference sequence.

The peptides (proteins containing one or more epitopes, epitopes, cassettes of epitopes and or TLR-binding proteins, or TLR-binding domains thereof) useful in the present invention can be optionally flanked and/or modified at one or both of the N- and C-termini, as desired, by amino acids from the naturally occurring sequences, amino acids added to facilitate linking to another peptide or to a lipid, other N- and C-terminal modifications, linked to carriers, etc., as described herein. Additional amino acids can be added to the termini of a peptide to provide for modifying the physical or chemical properties of the peptide or the like. Amino acids such as tyrosine, cysteine, lysine, glutamic or aspartic acid, or the like, can be introduced at the C- or N-terminus of the peptide or oligopeptide. In addition, the peptide sequences can differ from the natural sequence by being modified by terminal-NH₂ acylation, e.g., by alkanoyl (C₁-C₂₀) or thioglycolyl acetylation, terminal-carboxy amidation, e.g., ammonia, methylamine, etc. In some instances these modifications may provide sites for linking to a support or other molecule.

As described further below, the present invention also provides polynucleotides encoding the epitopes, polypeptides comprising epitopes, TLR-binding proteins, or TLR-binding domains thereof, and cassettes of such polypeptides as described herein.

### The Second Domain

The second domain of the immune response altering agents of the present invention comprises one or more heterologous target molecules. As described herein, the heterologous target molecules comprise any molecule against which it is desired to alter an immune response (e.g., either generate an immune response against or downregulate an existing aberrant immune response being mounted against the target molecule).

The term “heterologous” when used with reference to the target molecules of the present invention indicates a molecule, such as a protein, that generally is not found in the same relationship in nature to the epitopes, or cassettes of epitopes, of the first domain. For example, a fusion polypeptide comprising a first domain comprising a polypeptide or subsequence from different polypeptides, e.g., epitopes from multiple polypeptides, and/or TLR-binding proteins, or TLR-binding domains thereof, are fused to a heterologous protein or proteins that are not naturally in an adjacent position to the epitopes comprised in the first domain. Accordingly, a heterologous target molecule refers to any molecule, such as a protein, that is different from the T or B cell epitopes or TLR-binding proteins or TLR-binding domains thereof, present in the first domain of the present invention. Further, the heterologous target molecule may comprise any substance against which it is desirable to generate an immune response (e.g., polypeptide, polysaccharide, lipid, glycolipid, carbohydrate, lipopolysaccharide, etc.). Thus, as used herein, the heterologous target molecule may comprise a molecule that is not protein. In certain embodiments, the heterologous target molecule may comprise any non-proteinaceous organic molecule, chemical compound or moiety, or inorganic molecule.

Heterologous targets of the present invention include but are not limited to antigens derived from autoantigens common in autoimmune diseases such as rheumatoid arthritis, multiple sclerosis, insulin dependent diabetes, Addison's disease, celiac disease, chronic fatigue syndrome, inflammatory bowel disease, ulcerative colitis, Crohn's disease, Fibromyalgia, systemic lupus erythematosus, psoriasis, Sjogren's syndrome, hyperthyroidism/Graves disease, hypothyroidism/Hashimoto's disease, Insulin-dependent diabetes (type 1), Myasthenia Gravis, endometriosis, scleroderma, pernicious anemia, Goodpasture syndrome, Wegener's disease, glomerulonephritis, aplastic anemia, paroxysmal nocturnal hemoglobinuria, myelodysplastic syndrome, idiopathic thrombocytopenic purpura, autoimmune hemolytic anemia, Evan's syndrome, Factor VIII inhibitor syndrome, systemic vasculitis, dermatomyositis, polymyositis and rheumatic fever. Illustrative autoantigens of the present invention include but are not limited to, myelin basic protein (MBP), MBP 84-102, MBP 143-168, pancreatic islet cell antigens, collagen, CLIP-170, thyroid antigens, nucleic acid, acetylcholine receptor, S Antigen, and type II collagen.

Heterologous targets of the present invention also include proteins or other antigens derived from a variety of infectious agents. Infectious agents include but are not limited to, any variety of viruses, such as, single stranded RNA viruses, single stranded DNA viruses, cytomegalovirus (CMV), Rous sarcoma virus (RSV), hepatitis A virus, hepatitis B virus (HBV), Hepatitis C (HCV), Herpes viruses, such as herpes simplex virus (HSV), Influenza viruses, west nile virus (WNV), Epstein-Barr virus (EBV), eastern equine encephalitis virus (EEEV), severe acute respiratory virus (SARS), human immunodeficiency virus (HIV), human papilloma virus (HPV), and human T cell lymphoma virus (HTLV); parasites (e.g., protozoan and metazoan pathogens such as Plasmodia species, Leishmania species, Schistosoma species, Trypanosoma species, flagellated protozoa Giardia duodenalis; Entamoebae), bacteria (e.g., Mycobacteria, in particular, M. tuberculosis, Salmonella, Streptococci, E. coli, Staphylococci, Chlamydia species, Pseudomonads), fungi (e.g., Candida species, Aspergillus species), and Pneumocystis carinii. For example, heterologous targets may include viral coat proteins, i.e., influenza neuraminidase and hemmaglutinin, HIV gp 160 or derivatives thereof, SARS coat proteins, herpes virion proteins, WNV proteins, etc. Heterologous targets may also include bacterial surface proteins including pneumococcal PsaA, PspA, LytA, surface or virulence associated proteins of bacterial pathogens such as Nisseria gonnorhea, outer membran proteins or surface proteases.

Heterologous targets of the present invention include but are not limited to antigens derived from a variety of tumor proteins. Illustrative tumor proteins useful in the present invention include, but are not limited to any one or more of, p53, MAGE-A1, MAGE-A2, MAGE-A3, MAGE-A4, MAGE-A6, MAGE-A10, MAGE-A12, BAGE, DAM-6,-10, GAGE-1,-2,-8, GAGE-3,-4,-5,-6,-7B, NA88-A, NY-ESO-1, MART-1, MCIR, Gp100, PSA, PSM, Tyrosinase, TRP-1, TRP-2, ART-4, CAMEL, CEA, Cyp-B, Her2/neu, hTERT, hTRT, iCE, MUCd, MUC2, PRAME, P15, RUI, RU2, SART-1, SART-3, WT1, AFP, β-catenin/m, Caspase-8/m, CEA, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, ETV6/AML, LDLR/FUT, Pml/RARα, and TEL/AMLI. These and other tumor proteins are known to the skilled artisan.

In certain embodiments, tumor antigens may be identified directly from an individual with cancer. In this regard, screens can be carried out using a variety of known technologies. For example, in one embodiment, a tumor biopsy is taken from a patient, RNA is isolated from the tumor cells and screened using a gene chip (for example, from Affymetrix, Santa Clara, Calif.) and a tumor antigen is identified. Once the tumor target antigen is identified, it may then be cloned, expressed and purified using techniques known in the art. This target molecule is then linked to one or more epitopes/cassettes of the present invention as described herein and administered to the cancer patient in order to alter the immune response to the target molecule isolated from the tumor. In this manner, “personalized vaccines” are contemplated within the context of the invention.

Generally, the alteration in an immune response comprises an induction of a humoral response and/or a cellular response. As such “alteration” of an immune response comprises any statistically significant change, e.g. increase or decrease, in the level of one or more immune cells (T cells, B cells, antigen-presenting cells, dendritic cells, and the like) or in the activity of one or more of these immune cells (CTL activity, HTL activity, cytokine secretion, change in profile of cytokine secretion, etc.). The skilled artisan would readily appreciate that a number of methods for establishing whether an alteration in the immune response has taken place are available. A-variety of methods for detecting alterations in an immune response (e.g. cell numbers, cytokine expression, cell activity) are known in the art and are useful in the context of the instant invention. Illustrative methods are described in Current Protocols in Immunology, Edited by: John E. Coligan, Ada M. Kruisbeek, David H. Margulies, Ethan M. Shevach, Warren Strober (2001 John Wiley & Sons, NY, N.Y.) Ausubel et al. (2001 Current Protocols in Molecular Biology, Greene Publ. Assoc. Inc. & John Wiley & Sons, Inc., NY, N.Y.); Sambrook et al. (1989 Molecular Cloning, Second Ed., Cold Spring Harbor Laboratory, Plainview, N.Y.); Maniatis et al. (1982 Molecular Cloning, Cold Spring Harbor Laboratory, Plainview, N.Y.) and elsewhere. Illustrative methods useful in this context include intracellular cytokine staining (ICS), ELISPOT, proliferation assays, cytotoxic T cell assays including chromium release or equivalent assays, and gene expression analysis using any number of polymerase chain reaction (PCR) or RT-PCR based assays.

In certain embodiments, alteration of an immune response comprises an increase in heterologous target-specific CTL activity of between 1.5 and 5 fold in a subject administered the heterologous target molecule in the presence of one or more of the epitopes of the present invention as compared to in the absence of the epitopes of the present invention. In another embodiment, alteration of an immune response comprises an increase in heterologous target-specific CTL activity of about 2, 2.5, 3, 3.5, 4, 4.5, 5, 5.5, 6, 6.5, 7, 7.5, 8, 8.5, 9, 9.5, 10, 10.5, 11, 11.5, 12, 12.5, 15, 16, 17, 18, 19, 20, or more fold in a subject administered the heterologous target molecule in the presence of one or more of the epitopes of the present invention as compared to in the absence of the epitopes of the present invention.

In a further embodiment, alteration of an immune response comprises an increase in heterologous target-specific HTL activity, such as proliferation of helper T cells, of between 1.5 and 5 fold in a subject administered the heterologous target molecule in the presence of one or more of the epitopes of the present invention as compared to in the absence of the epitopes of the present invention. In another embodiment, alteration of an immune response comprises an increase in heterologous target-specific HTL activity of about 2, 2.5, 3, 3.5, 4, 4.5, 5, 5.5, 6, 6.5, 7, 7.5, 8, 8.5, 9, 9.5, 10, 10.5, 11, 11.5, 12, 12.5, 15, 16, 17, 18, 19, 20, or more fold in a subject administered the heterologous target molecule in the presence of one or more of the epitopes of the present invention as compared to in the absence of the epitopes of the present invention. In this context, an alteration in HTL activity may comprise an increase as described above, or decrease, in production of a particular cytokine, such as interferon-gamma (IFN-γ), interleukin-1 (IL-1), IL-2, IL-3, IL-6, IL-7, IL-12, IL-15, tumor necrosis factor-alpha (TNF-α), granulocyte macrophage colony-stimulating factor (GM-CSF), granulocyte -colony stimulating factor (G-CSF), or other cytokine. In this regard, an alteration in an immune response may comprise a shift from a Th2 type response to a Th1 type response or in certain embodiments a shift from a Th1 type response to a Th2 type response. In other embodiments, the alteration in an immune response may comprise the stimulation of a predominantly Th1 or a Th2 type response.

In a further embodiment, alteration of an immune response comprises an increase in heterologous target-specific antibody production of between 1.5 and 5 fold in a subject administered the heterologous target molecule in the presence of one or more of the epitopes of the present invention as compared to in the absence of the epitopes of the present invention. In another embodiment, alteration of an immune response comprises an increase in heterologous target-specific antibody production of about 2, 2.5, 3, 3.5, 4, 4.5, 5, 5.5, 6, 6.5, 7, 7.5, 8, 8.5, 9, 9.5, 10, 10.5, 11, 11.5, 12, 12.5, 15, 16, 17, 18, 19, 20, or more fold in a subject administered the heterologous target molecule in the presence of one or more of the epitopes of the present invention as compared to in the absence of the epitopes of the present invention.

### Coupling the First Domain to the Second Domain

In certain embodiments, the first domain is not fused to the second domain and rather, the domains are provided in an appropriate mixture or are administered separately. In this regard, the first domain to be administered in conjunction with the second domain may comprise a mixture of individual proteins (e.g., ESAT6, CFPlO, one or more TLR-binding protein, or other suitable proteins)/epitopes as described herein. As such, the first domain as described herein acts as a sort of adjuvant to modulate the immune response to the heterologous target. Without being bound by theory, the first domain, whether coupled, fused or otherwise attached to the second domain or not fused to the second domain, acts to recruit antigen presenting cells and other cells of the immune system (e.g., T cells, B cells, NK cells, and the like) to the site, allowing them to appropriately process antigen and thereby initiating an appropriate immune response (e.g., CD4⁺ or CD8⁺ T cell response or an antibody mediated response).

The present invention provides for a variety of mechanisms by which the first domain may be coupled, fused or otherwise attached to the first domain. Note that in certain embodiments, the first domain is not fused to the second domain and rather, the domains are provided in an appropriate mixture.

In one embodiment, the first domain and second domain are produced recombinantly as described herein. As such, the two domains are produced as a fusion protein and are covalently attached via a peptide bond. In certain embodiments, the two domains may be separated by one or more of a variety of linkers. In one embodiment the domains are linked via covalent linkages. The domains may be covalently linked by any suitable means, such as via a cross-linking reagent or a polypeptide linker.

In a further embodiment, the first domain is chemically coupled to the second domain. Chemical coupling may be achieved using commercially available homo- or hetero-bifunctional cross-linking compounds, according to methods known and available in the art, such as those described, for example, in Hermanson, Greg T., Bioconjugate Techniques, Academic. Press, Inc., 1995, and Wong, Shan S., Chemistry of Protein Conjugation and Cross-linking, CRC Press, 1991. As an example, when the terminal residues are cysteines, the coupling with lysine residues may be carried out by using SPDP (N-succinimidyl-3-(2-pyridylthio)-propionate, or sulfo-MBS described by Lerner et al. in (Nature, October 1980, p 801 to 805, vol. 287) or other bifunctional reagents. As a further example, when the coupling implicates tyrosine residues, other coupling reagents may be used. In yet another example, when the residues to be coupled are lysine residues, it will be possible to use glutaraldehyde.

In another embodiment, the first domain is noncovalently attached to the second domain, such as via an electrostatic interaction, a hydrophobic interaction or through the interaction of biotin and avidin or streptavidin. In certain embodiments, the two domains are attached via an antibody/ligand interaction. In further embodiments, the two domains are mechanically coupled, such as through interaction of filaments of actin and myosin, integrins, or other such proteins.

In a further embodiment of the present invention, functional groups are added to the first or second domain, or both, such that other compounds, such as lipopolysaccharide, polysaccharides, carbohydrates, lipids, and the like, can then be attached thereto. In this regard, any of a wide variety of functional groups are contemplated for use in the present invention, for example, such as those described by Lehninger, Nelson, and Cox, Principles of Biochemistry, 2nd Edition, Worth Publishers, 1993; Murray R., Mayes P. A., Rodwel V., Granner D., Harper's Biochemistry, 26th Ed., The McGraw-Hill Companies 2003. In certain embodiments, as noted elsewhere herein, the heterologous target molecule comprises a lipopolysaccharide, polysaccharides, carbohydrates, lipids, and the like, attached via a functional group to the first domain using any number of techniques known to the skilled artisan. In certain embodiments, the two domains are attached via UV crosslinking.

In certain embodiments, the first domain is produced and stored appropriately until needed and then linked or otherwise attached to a target molecule of interest using any of a variety of methods as described herein. In one embodiment, the first domain is contacted with a viral, bacterial, or tumor preparation in order to randomly couple or otherwise attach viral, bacterial, or tumor target proteins thereto.

In a further embodiment the immune response altering agents described herein (e.g., the first domain coupled to the second domain) are attached to a targeting molecule for targeting the immune response altering agent to a cell, tissue, or organ of interest. In this regard, the immune response altering agent may be attached or otherwise coupled to an antibody using methods known in the art and described herein (e.g., recombinantly or otherwise engineered, or using any method as described above for coupling the first and second domains, etc.). The targeting molecule is a molecule for which the desired cell, tissue or organ has a requirement or a receptor, as described herein. As such, targeting molecules include a molecule which is bound by a receptor and transported into a cell by a receptor-mediated process, or otherwise specifically taken up into a target cell, tissue, or organ of interest. Examples of suitable targeting molecules include, but are not limited to, glucose, galactose, mannose, mannose 6-phosphate, transferrin, asialoglycoprotein, alpha.-2-macroglobulins, insulin, a peptide growth factor, cobalamin, folic acid or derivatives, biotin or derivatives, YEE(GaINAcAH)₃ or derivatives thereof, albumin, texaphyrin, metallotexaphyrin, porphyrin, any vitamin, any coenzyme, an antibody, an antigen-binding fragment of an antibody (e.g., Fab) and a single chain antibody variable region (scFv). A skilled artisan will readily recognize other targeting molecules (ligands) which bind to cell receptors and which are transported into a cell by a receptor-mediated process. The present invention is intended to include all such targeting molecules.

### Polynucleotides Encoding Immune Response Altering Agents and/or Components Thereof

The present invention further provides polynucleotides that encode an immune response altering agent. As such, the present invention provides polynucleotides that encode an epitope, protein comprising. one or more epitopes, TLR-binding proteins, or TLR-binding domains thereof, other components (e.g., MHC targeting sequences, LAMP-1, and the like), or heterologous target proteins described herein, expression vectors comprising such polynucleotides and host cells transformed or transfected with such expression vectors. The terms “DNA” and “polynucleotide” are used essentially interchangeably herein to refer to a DNA molecule that has been isolated free of total genomic DNA of a particular species. An isolated polynucleotide, as used herein, means that a polynucleotide is substantially away from other coding sequences, and that the DNA molecule does not contain large portions of unrelated coding DNA, such as large chromosomal fragments or other functional genes or polypeptide coding regions. Of course, this refers to the DNA molecule as originally isolated, and does not exclude genes or coding regions later added to the segment by the hand of man.

As will be understood by those skilled in the art, the polynucleotides of this invention can include genomic sequences, extra-genomic and plasmid-encoded sequences and smaller engineered gene segments that express, or may be adapted to express epitopes as described herein, proteins containing epitopes, polypeptides, peptides and the like. Such segments may be naturally isolated, or modified synthetically by the hand of man.

As will be also recognized by the skilled artisan, polynucleotides of the invention may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes a polypeptide/protein/epitope of the invention or a portion thereof) or may comprise a sequence that encodes a variant or derivative of such a sequence. In certain preferred embodiments, the polynucleotide sequences set forth herein encode proteins comprising epitopes, epitopes, cassettes of epitopes, or heterologous target proteins as described herein.

In other related embodiments, the present invention provides polynucleotide variants having substantial identity to native sequences encoding epitopes or proteins comprising epitopes or heterologous target proteins as described herein, for example those comprising at least 70% sequence identity, preferably at least 75%, 80%, 85%, 90%, 95%, 96%, 97%, 98%, or 99% or higher, sequence identity compared to a native polynucleotide sequence encoding the polypeptides of this invention using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

Typically, polynucleotide variants will contain one or more substitutions, additions, deletions and/or insertions, preferably such that the immunogenicity of the epitope of the polypeptide encoded by the variant polynucleotide or such that the immunogenicity of the heterologous target protein is not substantially diminished relative to a polypeptide encoded by the native polynucleotide sequence. As described elsewhere herein, the polynucleotide variants preferably encode an epitope variant wherein the ability of the variant epitope to react with antigen-specific antisera and/or T-cell lines or clones is not substantially diminished relative to the native polypeptide. The term “variants” should also be understood to encompasses homologous genes of xenogenic origin.

The present invention provides polynucleotides that comprise or consist of at least about 10, 15, 20, 30, 40, 50, 75, 100, 150, 200, 300, 400, 500 or 1000 or more contiguous nucleotides encoding a polypeptide, including heterologous target proteins, as described herein, as well as all intermediate lengths there between. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like. A polynucleotide sequence as described here may be extended at one or both ends by additional nucleotides not found in the native sequence encoding a polypeptide as described herein, such as an epitope or heterologous target protein. This additional sequence may consist of 1, 2, 3, 4, 5, 6, 7, 8, 9, 10, 11, 12, 13, 14, 15, 16, 17, 18, 19, or 20 nucleotides or more, at either end of the disclosed sequence or at both ends of the disclosed sequence.

The polynucleotides of the present invention, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative polynucleotide segments with total lengths of about 10,000, about 5000, about 3000, about 2,000, about 1,000, about 500, about 200, about 100, about 50 base pairs in length, and the like, (including all intermediate lengths) are contemplated to be useful in many implementations of this invention.

When comparing polynucleotide sequences, two sequences are said to be “identical” if the sequence of nucleotides in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., Unified Approach to Alignment and Phylogenes, pp. 626-645 (1990); Methods in Enzymology vol. 183, Academic Press, Inc., San Diego, Calif.; Higgins, D. G. and Sharp, P. M., CABIOS 5:151-153 (1989); Myers, E. W. and Muller W., CABIOS 4:11-17 (1988); Robinson, E. D., Comb. Theor 11:105 (1971); Saitou, N. Nei, M., Mol. Biol. Evol. 4:406-425 (1987); Sneath, P. H. A. and Sokal, R. R., Numerical Taxonomy—the Principles and Practice of Numerical Taxonomy, Freeman Press, San Francisco, Calif. (1973); Wilbur, W. J. and Lipman, D. J., Proc. Natl. Acad., Sci. USA 80:726-730 (1983).

Alternatively, optimal alignment of sequences for comparison may be conducted by the local identity algorithm of Smith and Waterman, Add. APL. Math 2:482 (1981), by the identity alignment algorithm of Needleman and Wunsch, J. Mol. Biol. 48:443 (1970), by the search for similarity methods of Pearson and Lipman, Proc. Natl. Acad. Sci. USA 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al., Nucl. Acids Res. 25:3389-3402 (1977), and Altschul et al., J. Mol. Biol. 215:403-410 (1990), respectively. BLAST and BLAST 2.0 can be used, for example with the parameters described herein, to determine percent sequence identity for the polynucleotides of the invention. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a word length (W) of 11, and expectation (E) of 10, and the BLOSUM62 scoring matrix (see Henikoff and Henikoff, Proc. Natl. Acad. Sci. USA 89:10915 (1989)) alignments, (B) of 50, expectation (E) of 10, M=5, N=−4 and a comparison of both strands.

Preferably, the “percentage of sequence identity” is determined by comparing two optimally aligned sequences over a window of comparison of at least 20 positions, Wherein the portion of the polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases occurs in both sequences to yield the number of matched positions, dividing the number of matched positions by the total number of positions in the reference sequence (i.e., the window size) and multiplying the results by 100 to yield the percentage of sequence identity.

It will be appreciated by those of ordinary skill in the art that, as a result of the degeneracy of the genetic code, there are many nucleotide sequences that encode an epitope, polypeptide comprising an epitope, or heterologous target protein, as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Therefore, in another embodiment of the invention, a mutagenesis approach, such as site-specific mutagenesis, is employed for the preparation of variants and/or derivatives of the epitopes or polypeptides comprising the epitopes as described herein. By this approach, specific modifications in a polypeptide sequence can be made through mutagenesis of the underlying polynucleotides that encode them. These techniques provides a straightforward approach to prepare and test sequence variants, for example, incorporating one or more of the foregoing considerations, by introducing one or more nucleotide sequence changes into the polynucleotide.

Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

In certain embodiments of the present invention, the inventors contemplate the mutagenesis of the disclosed polynucleotide sequences to alter one or more properties of the encoded polypeptide, such as the immunogenicity of an epitope comprised in a polypeptide. The techniques of site-specific mutagenesis are well known in the art, and are widely used to create variants of both polypeptides and polynucleotides. For example, site-specific mutagenesis is often used to alter a specific portion of a DNA molecule. In such embodiments, a primer comprising typically about 14 to about 25 nucleotides or so in length is employed, with about 5 to about 10 residues on both sides of the junction of the sequence being altered.

The preparation of sequence variants of the selected peptide-encoding DNA segments using site-directed mutagenesis provides a means of producing potentially useful species and is not meant to be limiting, as there are other ways in which sequence variants of peptides and the DNA sequences encoding them may be obtained. For example, recombinant vectors encoding the desired peptide sequence may be treated with mutagenic agents, such as hydroxylamine, to obtain sequence variants. Specific details regarding these methods and protocols are found in the teachings of Maloy et al., 1994; Segal, 1976; Prokop and Bajpai, 1991; Kuby, 1994; and Maniatis et al., 1982.

Polynucleotide segments or fragments encoding the polypeptides of the present invention may be readily prepared by, for example, directly synthesizing the fragment by chemical means, as is commonly practiced using an automated oligonucleotide synthesizer. Also, fragments may be obtained by application of nucleic acid reproduction technology, such as the PCR™ technology of U.S. Pat. No. 4,683,202, by introducing selected sequences into recombinant vectors for recombinant production, and by other recombinant DNA techniques generally known to those of skill in the art of molecular biology (see for example, Current Protocols in Molecular Biology, John Wiley and Sons, NY, N.Y.).

In order to express a desired epitope, polypeptide comprising an epitope, cassette of epitopes, heterologous target protein, or fusion protein comprising any of the above, as described herein, the nucleotide sequences encoding the polypeptide, or functional equivalents, may be inserted into appropriate expression vector, i.e., a vector which contains the necessary elements for the transcription and translation of the inserted coding sequence and any desired linkers. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described, for example, in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York. N.Y.

A variety of expression vector/host systems may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

The “control elements” or “regulatory sequences” present in an expression vector are those non-translated regions of the vector—enhancers, promoters, 5′ and 3′ untranslated regions-which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the pBLUESCRIPT phagemid (Stratagene, La Jolla, Calif.) or pSPORTI plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, any of a number of expression vectors may be selected depending upon the use intended for the expressed polypeptide. For example, when large quantities are needed, for example for the induction of antibodies, vectors which direct high level expression of fusion proteins that are readily purified may be used. Such vectors include, but are not limited to, the multifunctional E. coli cloning and expression vectors such as pBLUESCRIPT (Stratagene), in which the sequence encoding the polypeptide of interest may be ligated into the vector in frame with sequences for the amino-terminal Met and the subsequent 7 residues of .beta.-galactosidase so that a hybrid protein is produced; pIN vectors (Van Heeke, G. and S. M. Schuster (1989) J. Biol. Chem. 264:5503-5509); and the like. pGEX Vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH may be used. For reviews, see Ausubel et al. (supra) and Grant et al. (1987) Methods Enzymol. 153:516-544.

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, N. (1987) EMBO J. 6:307-311. Alternatively, plant promoters such as the small subunit of RUBISCO or heat shock promoters may be used (Coruzzi, G. et al. (1984) EMBO J. 3:1671-1680; Broglie, R. et al. (1,984) Science 224:838-843; and Winter, J. et al. (1991) Results Probl. Cell Differ. 17:85-105). These constructs can be introduced into plant cells by direct DNA transformation or pathogen-mediated transfection. Such techniques are described in a number of generally available reviews (see, for example, Hobbs, S. or Murry, L. E. in McGraw Hill Yearbook of Science and Technology (1992) McGraw Hill, New York, N.Y.; pp. 191-196).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia larvae. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. fiugiperda cells or Trichoplusia larvae in which the polypeptide of interest may be expressed (Engelhard, E. K. et al. (1994) Proc. Natl. Acad. Sci. 91:3224-3227).

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential El or E3 region of the viral genome may be used to obtain a viable virus which is capable of expressing the polypeptide in infected host cells (Logan, J. and Shenk, T. (1984) Proc. Natl. Acad. Sci. 81:3655-3659). In addition, transcription enhancers, such as the Rous sarcoma virus (RSV) enhancer, may be used to increase expression in mammalian host cells.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf, D. et al. (1994) Results Probl. Cell Differ. 20:125-162).

In addition, a host cell strain may be chosen for its ability to modulate the expression of the inserted sequences or to process the expressed protein in the desired fashion. Such modifications of the polypeptide include, but are not limited to, acetylation, carboxylation. glycosylation, phosphorylation, lipidation, and acylation. Post-translational processing which cleaves a “prepro” form of the protein may also be used to facilitate correct insertion, folding and/or function. Different host cells such as CHO, COS, HeLa, MDCK, HEK293, and W138, which have specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a polynucleotide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler, M. et al. (1977) Cell 11:223-32) and adenine phosphoribosyltransferase (Lowy, I. et al. (1990) Cell 22:817-23) genes which can be employed in tk⁻ or aprt⁻ cells, respectively. Also, ant metabolite, antibiotic or herbicide resistance can be used as the basis for selection; for example, defer which confers resistance to methotrexate (Wigler, M. et al. (1980) Proc. Natl. Acad. Sci. 77:3567-70); npt, which confers resistance to the aminoglycosides, neomycin and G-418 (Colbere-Garapin, F. et al (1981) J. Mol. Biol. 150:1-14); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman, S. C. and R. C. Mulligan (1988) Proc. Natl. Acad. Sci. 85:8047-51). The use of visible markers has gained popularity with such markers as anthocyanins, beta-glucuronidase and its substrate GUS, and luciferase and its substrate luciferin, being widely used not only to identify transformants, but also to quantify the amount of transient or stable protein expression attributable to a specific vector system (Rhodes, C. A. et al. (1995) Methods Mol. Biol. 55:121-131).

Although the presence/absence of marker gene expression suggests that the gene of interest is also present, its presence and expression may need to be confirmed. For example, if the sequence encoding a polypeptide is inserted within a marker gene sequence, recombinant cells containing sequences can be identified by the absence of marker gene function. Alternatively, a marker gene can be placed in tandem with a polypeptide-encoding sequence under the control of a single promoter. Expression of the marker gene in response to induction or selection usually indicates expression of the tandem gene as well.

Alternatively, host cells that contain and express a desired polynucleotide sequence may be identified by a variety of procedures known to those of skill in the art. These procedures include, but are not limited to, DNA-DNA or DNA-RNA hybridizations and protein bioassay or immunoassay techniques which include, for example, membrane, solution, or chip based technologies for the detection and/or quantification of nucleic acid or protein.

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on a given polypeptide may be preferred for some applications, but a competitive binding assay may also be employed. These and other assays are described, among other places, in Hampton, R. et al. (1990; Serological Methods, a Laboratory Manual, APS Press, St Paul. Minn.) and Maddox, D. E. et al. (1983; J. Exp. Med. 158:1211-1216).

In addition to recombinant production methods, polypeptides of the invention, and fragments thereof, may be produced by direct peptide synthesis using solid-phase techniques (Merrifield J. (1963) J. Am. Chem. Soc. 85:2149-2154). Protein synthesis may be performed using manual techniques or by automation. Automated synthesis may be achieved, for example, using Applied Biosystems 431A Peptide Synthesizer (Perkin Elmer). Alternatively, various fragments may be chemically synthesized separately and combined using chemical methods to produce the full-length molecule.

### Methods of Altering an Immune Response

The invention provides methods of activating an immune response in which cells of the immune system are exposed to one or more immune altering agents of the invention. The methods of the invention can be performed in vitro, in vivo, or ex vivo. In vitro application of the immune response altering agents can be useful, for example, in basic scientific studies of immune mechanisms or for production of activated T or B cells for use in either studies on T cell or B cell function or, for example, adoptive immunotherapy.

### In Vitro Methods

In the in vitro methods of the invention, T cells (CD4+ and/or CD8+) obtained from a mammalian subject (see below) are cultured with an immune response altering agent of the invention and antigen presenting cells (APC), preferably, but not necessarily, obtained from the same individual as the T cells. Where the APC are obtained from a different individual, the donor of the T cells and the donor of the APC will preferably express at least one major histocompatibility complex (MHC) molecule (e.g., a MHC class II molecule) in common. APC can be essentially any MHC expressing cell. In certain embodiments, they will be MHC class II-expressing cells. Thus, they can be, for example, interdigitating dendritic cells (DC), macrophages, monocytes, B cells, or cell lines (clonal or non-clonal) derived from any of these cells. They can also be any cell type (e.g., fibroblasts) transfected or transduced with and expressing a polynucleotide encoding an MHC class II molecule. Such cultures can also be supplemented with one or more cytokines or growth factors such as, without limitation, IL-1, IL-2, IL-3, IL-6, IL-7, IL-12, IL-15, IFN-γ, TNF-α, granulocyte macrophage colony-stimulating factor (GM-CSF), or granulocyte-colony stimulating factor (G-CSF). The cultures can be “restimulated” as often as necessary with either the immune response altering agent or either of the domains thereof alone. The cultures can also be monitored at various times to ascertain whether the desired alteration in the immune response has been attained (e.g., an increase in CTL activity, increased cytokine production, increased proliferation, etc.).

### In Vivo Methods

In one in vivo approach, the immune response altering agent is administered to the subject. Generally, the immune response altering agents of the invention will be suspended in a pharmaceutically-acceptable carrier (e.g., physiological saline) and administered orally or by intravenous infusion, or injected subcutaneously, intramuscularly, intraperitoneally, intrarectally, intravaginally, intranasally, intragastrically, intratracheally, or intrapulmonarily. In certain embodiments, the immune response altering agents are delivered directly to an appropriate lymphoid tissue (e.g. spleen, lymph node, or mucosal-associated lymphoid tissue (MALT)). The dosage required depends on the choice of the route of administration, the nature of the formulation, the nature of the patient's illness, the subject's size, weight, surface area, age, and sex, other drugs being administered, and the judgment of the attending physician. Suitable dosages are in the range of 0.01-100.00 μg/kg. Wide variations in the needed dosage are to be expected in view of the variety of immune response altering agents available and the differing efficiencies of various routes of administration. For example, oral administration would be expected to require higher dosages than administration by i.v. injection. Variations in these dosage levels can be adjusted using standard empirical routines for optimization as is well understood in the art. Administrations can be single or multiple (e.g., 2- or 3-, 4-, 6-, 8-, 10-, 20-, 50-, 100-, 150-, or more fold). Encapsulation of the polypeptide in a suitable delivery vehicle (e.g., polymeric microparticles or implantable devices) may increase the efficiency of delivery, particularly for oral delivery.

Alternatively, a polynucleotide containing a nucleic acid sequence encoding a protein or a fusion protein of interest can be delivered to an appropriate cells of the animal. Expression of the coding sequence will preferably be directed to lymphoid tissue of the subject by, for example, delivery of the polynucleotide to the lymphoid tissue. This can be achieved by, for example, the use of a polymeric, biodegradable mnicroparticle or microcapsule delivery vehicle, sized to optimize phagocytosis by phagocytic cells such as macrophages. For example, PLGA (poly-lacto-co-glycolide) microparticles approximately 1-10 μm in diameter can be used. The polynucleotide is encapsulated in these microparticles, which are taken up by macrophages and gradually biodegraded within the cell, thereby releasing the polynucleotide. Once released, the DNA is expressed within the cell. A second type of microparticle is intended not to be taken up directly by cells, but rather to serve primarily as a slow-release reservoir of nucleic acid that is taken up by cells only upon release from the micro-particle through biodegradation. These polymeric particles should therefore be large enough to preclude phagocytosis (i.e., larger than 5 μm and preferably larger than 20 μm).

Another way to achieve uptake of the nucleic acid is using liposomes, prepared by standard methods. The vectors can be incorporated alone into these delivery vehicles or co-incorporated with tissue-specific antibodies. Alternatively, one can prepare a molecular conjugate composed of a plasmid or other vector attached to poly-L-lysine by electrostatic or covalent forces. Poly-L-lysine binds to a ligand that can bind to a receptor on target cells [Cristiano et al. (1995), J. Mol. Med. 73, 479]. Alternatively, lymphoid tissue specific targeting can be achieved by the use of lymphoid tissue-specific transcriptional regulatory elements (TRE) such as a B lymphocyte, T lymphocyte, or dendritic cell specific TRE. Lymphoid tissue specific TRE are known [Thompson et al. (1992), Mol. Cell. Biol. 12, 1043-1053; Todd et al. (1993), J. Exp. Med. 177, 1663-1674; Penix et al. (1993), J. Exp. Med. 178, 1483-1496]. Delivery of “naked DNA” (i.e., without a delivery vehicle) to an intramuscular, intradermal, or subcutaneous site, is another means to achieve in vivo expression.

In the relevant polynucleotides (e.g., expression vectors) the nucleic acid sequence encoding the fusion protein of interest with an initiator methionine and optionally a targeting sequence is operatively linked to a promoter or enhancer-promoter combination.

Polynucleotides can be administered in a pharmaceutically acceptable carrier. Pharmaceutically acceptable carriers are biologically compatible vehicles which are suitable for administration to a human or other mammalian subject, e.g., physiological saline. A therapeutically effective amount is an amount of the polynucleotide which is capable of producing a medically desirable result (e.g., an enhanced T cell response) in a treated animal. As is well known in the medical arts, the dosage for any one patient depends upon many factors, including the patient's size, body surface area, age, the particular compound to be administered, sex, time and route of administration, general health, and other drugs being administered concurrently. Dosages will vary, but a preferred dosage for administration of polynucleotide is from approximately 10⁶ to 10¹² copies of the polynucleotide molecule. This can be repeatedly administered, as needed. Routes of administration can be any of those listed above.

### Ex Vivo Methods

In one ex vivo approach, lymphoid cells, including T cells (CD4+ and/or CD8+ T cells), and/or APC, are isolated from the subject and exposed to the immune response altering agent in vitro (see above). The lymphoid cells can be exposed once or multiply (e.g., 2, 3, 4, 6, 8, or 10 times). The pattern of cytokine production by the lymphoid cells, or other measure of cell activity, such as CTL activity, can be tested after one or more exposures. Once the desired cytokines are being produced by the lymphoid cells, or other desired effector functions are displayed by the cells, they are reintroduced into the subject via any of the routes listed herein. The therapeutic or prophylactic efficacy of this ex vivo approach is dependent on the ability of the ex vivo activated lymphocytes to either: (a) exert, directly or indirectly, a neutralizing or cytotoxic effect on, for example, infectious microorganisms, host cells infected with microorganisms, or tumor cells; or (b) actively suppress a pathogenic T cell response as, for example, in SLE, MG, or other autoimmune diseases described herein.

It can generally be stated that a composition comprising the subject T or B cells, or activated APC, such as macrophages, may be administered at a dosage of 10⁴ to 10⁷ cells/kg body weight, preferably 10⁵ to 10⁶ cells/kg body weight, including all integer values within those ranges. Cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain adoptive immunotherapy studies, T cells are administered approximately at 1×10⁹ to 2×10¹¹ cells to the patient. (See, e.g., U.S. Pat. No. 5,057,423). In some aspects of the present invention, particularly in the use of allogeneic or xenogeneic cells, lower numbers of cells, in the range of 10⁶/kilogram (10⁶-10¹¹ per patient) may be administered. In certain embodiments, T or B cells are administered at 1×10⁵, 1×10⁶, 1×10⁷, 1×10⁸, 5×10⁸, 1×10⁹, 5×10⁹, 1×10¹⁰, 5×10¹⁰, 1×10¹¹, 5×10¹¹, or 1×10¹² cells to the subject. T or B cell compositions may be administered multiple times at dosages within these ranges. The T or B cells may be autologous or heterologous (allogeneic or xenogeneic) to the patient undergoing therapy. If desired, the treatment may also include administration of mitogens (e.g., PHA) or lymphokines, cytokines, and/or chemokines (e,g., GM-CSF, IL-4, IL-13, Flt3-L, RANTES, MIP1α, etc.) as described herein to further enhance the immune response.

An alternative ex vivo strategy can involve transfecting or transducing cells obtained from the subject with a polynucleotide sequence encoding an immune response altering agent. The transfected or transduced cells are then returned to the subject. While such cells would preferably be lymphoid cells, they could also be any of a wide range of types including, without limitation, fibroblasts, bone marrow cells, macrophages, monocytes, dendritic cells, epithelial cells, endothelial cells, keratinocytes, or muscle cells in which they act as a source of the fusion protein for as long as they survive in the subject. The use of lymphoid cells would be particularly advantageous in that such cells would be expected to home to lymphoid tissue (e.g., lymph nodes or spleen) and thus the immune response altering agent would be produced in high concentration at the site where they exert their effect, i.e., activation of an immune response. By using this approach, as in to the above-described in vivo approach using fusion protein-encoding polynucleotides, active in vivo immunization with the fusion protein is achieved. The same genetic constructs and signal sequences described for the in vivo approach can be used for this ex vivo strategy.

The ex vivo methods include the steps of harvesting cells from a subject, culturing the cells, transducing them with an expression vector, and maintaining the cells under conditions suitable for expression of the fusion protein. These methods are known in the art of molecular biology. The transduction step is accomplished by any standard means used for ex vivo gene therapy, including calcium phosphate, lipofection, electroporation, viral infection, and biolistic gene transfer. Alternatively, liposomes or polymeric microparticles can be used. Cells that have been successfully transduced are then selected, for example, for expression of the desired protein (immune response altering agent) or of a drug resistance gene. The cells may then be lethally irradiated (if desired) and injected or implanted into the patient.

These methods of the invention can be applied to any of the diseases and species listed here. Methods to test whether an immune response altering agent is therapeutic for or prophylactic against a particular disease are known in the art. Where a therapeutic effect is being tested, a test population displaying symptoms of the disease (e.g., cancer patients) is treated with a test immune response altering agent, using any of the above-described strategies. A control population, also displaying symptoms of the disease, is treated, using the same methodology, with a placebo. Disappearance or a decrease of the disease symptoms in the test subjects would indicate that the immune response altering agent was an effective therapeutic agent.

By applying the same strategies to subjects prior to onset of disease symptoms (e.g., presymptomatic subjects considered to likely candidates for SLE development or candidates predisposed to a particular cancer or. experimental animals in which an appropriate disease spontaneously arises, e.g., NZB mice, or can be deliberately induced, e.g., multiple murine cancers), immune response altering agents can be tested for efficacy as prophylactic agents, i.e., vaccines. In this situation, prevention of onset of disease symptoms is tested.

### Compositions Comprising Immune Response Altering Agents

The present invention provides compositions (including pharmaceutical compositions) comprising an effective amount of a purified immune response agent and a suitable diluent, physiologically acceptable excipient, or carrier. Immune response agents administered in vivo preferably are in the form of a pharmaceutical composition.

The compositions of the present invention may contain an immune response altering agent in any form described herein, including oligomers, variants, derivatives, and biologically active fragments. In one embodiment of the invention, the composition comprises a soluble fusion protein comprising an immune response altering agent.

Immune response altering agents may be formulated according to known methods that are used to prepare pharmaceutically useful compositions. Components that are commonly employed in pharmaceutical formulations include those described in Remington's Pharmaceutical Sciences, 16th ed., 1980, Mack Publishing Company.

Immune response altering agents employed in a pharmaceutical composition preferably is purified such that the agent is substantially free of other proteins of natural or endogenous origin, desirably containing less than about 1% by mass of protein contaminants residual of production processes. Such compositions, however, can contain other proteins added as stabilizers, carriers, excipients or co-therapeutics.

Components of the compositions will be nontoxic to patients at the dosages and concentrations employed. Ordinarily, the preparation of such compositions entails combining an immune response altering agent or derivative thereof with buffers, antioxidants such as ascorbic acid, low molecular weight (less than about 10 residues) peptides, proteins, amino acids, carbohydrates including glucose, sucrose, or dextrans, chelating agents such as EDTA, glutathione, or other stabilizers and excipients. Neutral buffered saline is one appropriate diluent.

For therapeutic use, the compositions are administered in a manner and dosage appropriate to the indication and the patient. Administration may be by any suitable route, including but not limited to continuous infuision, local administration, sustained release from implants (gels, membranes, and the like), or intravenous injection.

The compositions of the present invention may be administered in conjunction with other therapeutic modalities known in the art, chemotherapy, radiation, immunosuppressive agents. The compositions of the present invention may be administered in conjunction with any of a variety of adjuvants or cytokines known in the art. Such composition preparation is generally described in, for example, M. F. Powell and M. J. Newman, eds., “Vaccine Design (the subunit and adjuvant approach),” Plenum Press (NY, 1995).

### Methods of Use

The immune response altering agents of the present invention and compositions thereof are useful for administration to mammals, particularly humans, to treat and/or prevent any disease for which an alteration in an immune response is desired. As such, the compositions of the present invention are useful to treat and/or prevent a variety of infectious and autoimmune diseases and cancers. Examples of diseases which can be treated using the immune response altering agents of the invention include disease caused by any variety of viruses, such as, single stranded RNA viruses, single stranded DNA viruses, cytomegalovirus (CMV), Rous sarcoma virus (RSV), hepatitis A virus, hepatitis B virus (HBV), Hepatitis C (HCV), Herpes viruses, such as herpes simplex virus (HSV), Influenza viruses, west nile virus (WNV), Epstein-Barr virus (EBV), eastern equine encephalitis virus (EEEV), severe acute respiratory virus (SARS), human immunodeficiency virus (HIV), human papilloma virus (HPV), and human T cell lymphoma virus (HTLV); parasites (e.g., protozoan and metazoan pathogens such as Plasmodia species, Leishmania species, Schistosoma species, Trypanosoma species, flagellated protozoa Giardia duodenalis; Entamoebae), bacteria (e.g., eubacterial genera Acholeplasma, Anaeroplasma, Asteroleplasma, Mycoplasma, Mycobacteria, in particular, M. tuberculosis, Salmonella, Streptococci, E. coli, Staphylococci, Chlamydia species, Pseudomonads), fungi (e.g., Candida species, Aspergillus species), and Pneumocystis carinii.

The immune response altering agents of the present invention and compositions thereof can be used to treat and/or prevent autoimmune diseases such as rheumatoid arthritis, multiple sclerosis, insulin dependent-diabetes, Addison's disease, celiac disease, chronic fatigue syndrome, inflammatory bowel disease, ulcerativecolitis, Crohn's disease, Fibromyalgia, systemic lupus erythematosus, psoriasis, Sjogren's syndrome, hyperthyroidism/Graves disease, hypothyroidism/Hashimoto's disease, Insulin-dependent diabetes (type 1), Myasthenia Gravis, endometriosis, scleroderma, pernicious anemia, Goodpasture syndrome, Wegener's disease, glomerulonephritis, aplastic anemia, paroxysmal nocturnal hemoglobinuria, myelodysplastic syndrome, idiopathic thrombocytopenic purpura, autoimmune hemolytic anemia, Evan's syndrome, Factor VIII inhibitor syndrome, systemic vasculitis, dermatomyositis, polymyositis and rheumatic fever.

Further uses of the compositions of the present invention may include the treatment and/or prophylaxis of: inflammatory and hyperproliferative skin diseases and cutaneous manifestations of immunologically mediated illnesses, such as, seborrhoeis dermatitis, angioedemas, erythemas, acne, and Alopecia areata; various eye diseases (autoimmune and otherwise); allergic reactions, such as pollen allergies, reversible obstructive airway disease, which includes condition such as asthma (for example, bronchial asthma, allergic asthma, intrinsic asthma, extrinsic asthma and dust asthma), particularly chronic or inveterate asthma (for example, late asthma and airway hyper-responsiveness), bronchitis, allergic rhinitis, and the like; inflammation of mucous and blood vessels.

The immune response altering agents of the invention can be used for treatment of disease conditions characterized by immunosuppression: e.g. cancer, AIDS or AIDS-related complex, other virally or environmentally-induced conditions, and certain congenital immune deficiencies. The compositions may also be employed to increase immune function that has been impaired by the use of radiotherapy of immunosuppressive drugs such as certain chemotherapeutic agents, and therefore are particularly useful when given in conjunction with such drugs or radiotherapy.

The immune response altering agents of the present invention and compositions thereof can be used to treat and/or prevent any of a variety of cancers, such as breast cancer, prostate cancer, colo-rectal cancer, kidney cancer, renal cell carcinoma, pancreatic cancer, esophageal cancer, brain cancer, lung cancer, ovarian cancer, cervical cancer, melanoma, non-Hodgkin's lymphoma, Hodgkin's disease, leukemia, plasmocytoma, sarcoma, glioma, thymoma, multiple myeloma, hepatocellular carcinoma, nasopharyngeal carcinoma, ALL, AML, CML, CLL, and other neoplasms known in the art.

The methods of the invention can be applied to a wide range of species, e.g., humans, non-human primates, horses, cattle, pigs, sheep, goats, dogs, cats, rabbits, guinea pigs, hamsters, rats, and mice.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety. Moreover, all numerical ranges utilized herein explicitly include all integer values within the range and selection of specific numerical values within the range is contemplated depending on the particular use. Further, the following examples are offered by way of illustration, and not by way of limitation.

From the foregoing it will be appreciated that, although specific embodiments of the invention have been described herein for purposes of illustration, various modifications may be made without deviating from the spirit and scope of the invention. Accordingly, the invention is not limited except as by the appended claims.

### EXAMPLE 1

### In Vivo Validation of Immune Response Altering Agents

Once the first domain elements are identified using any number of assays as described herein and known in the art, they are further validated in vivo.

An appropriate heterologous target molecule is chosen for testing and fused or otherwised attached to the first domain using, for example, recombinant DNA technology.

In vivo testing is carried out using appropriate mice as follows: Six to eight week old mycoplasma-free female BALB/c mice are immunized in groups of 6 with 100 μg of endotoxin free plasmid DNA in normal saline on day 0. Plasmids are injected intramuscularly into the thigh muscle of mice primed two days previously with 100 μl of 0.25% Bupivacaine. The identical booster dose on day 21 follows another similarly timed bupivacaine priming. The control mice are vaccinated with the vector plasmids not containing expressible inserts. All animal studies are conducted according to the appropriate animal care and use guidelines.

Estimation of antigen-specific cytoklie release. At necropsy on days 32 and 42, the mice are euthanized using CO₂ and exsanguinated by cardiac puncture. Spleens are removed aseptically using sterile instruments and transferred to a petri dish containing 5 ml of RPMI medium. Each excised spleen is dispersed into a single cell suspension, and the cells are washed once in RPMI medium and resuspended in complete RPMI (RPMI plus 10% fetal bovine serum and 5×10⁵ M beta-mercaptoethanol). For cytokine analysis, cell cultures are set up in Costar 48 well tissue culture clusters with 5 million cells per well containing 10 μg per ml of purified recombinant protein. The negative control wells have cells alone in complete RPMI while the positive control wells are stimulated with 1 μg per ml of Concanavalin A. The supernatants are collected after 72 hours and stored at −70° C.

The IFN-γ, IL-4 and IL-10 levels in culture supernatants are measured by a sandwich ELISA using a modified cytokine ELISA protocol. Ninety-six well plates are coated with capture antibody [for example, rat Mab XMG 1.2 for IFN-γ; rat Mab JES5-2A5 for IL-10; rat Mab 11B11 for IL-4 (PharMingen)] at 2 μg per ml in coating buffer overnight at 4° C., blocked with 1% gelatin for one hour at 37° C., and then incubated with 100 μl of culture supernatant in triplicate wells diluted either 1:2 or 1:4 in basal RPMJ for one hour at 37° C. For IFN-γ estimation, the plates are then incubated with 100 μl of a 1:2,000 dilution of rabbit anti-mouse IFN-γ antibodies followed by incubation with 100 μl of alkaline phosphatase-conjugated donkey anti-rabbit Immunoglobulin. Plates are incubated with biotinylated rat anti-mouse IL-10 (SXC-1) or biotinylated rat anti-mouse IL-4 (BVD6-24G2) (PharMingen) at 1:2,000 dilution followed by alkaline phosphatase-conjugated strepavidin. The plates are washed with PBS-Tween after each incubation step. The plates are developed by the addition of alkaline phosphatase substrate and the developed color quantified by measuring the OD at 405 nm. The concentrations of IFN-γ, IL-4 and IL-10 in the culture supernatants are estimated from the standard curves generated using mouse rIFN-γ, rIL-4 and rIL-10 (PharMingen) for the average of duplicate wells.

### Immunization with Recombinant Protein and In Vitro Assays for Cytokine Production

Groups of 6 mycoplasma-free female BALB/c mice are immunized with purified proteins according to the following schedule. Equal molar amounts of protein are injected intramuscularly on day 0 and boosted with an identical dose on day 14. Three mice from each group (test protein, negative control) are sacrificed on day 25 and three on day 35. The purified proteins are suspended in saline. The protein concentration is adjusted to provide an individual dose in 100 μl and is administered intramuscularly into the thigh muscle. The negative control mice are injected with normal saline. At necropsy, the mice are euthanized using CO₂ and exsanguinated by cardiac puncture. The blood is transferred into microcentrifuge tubes and allowed to clot at 4° C. Serum is collected the next day by centrifugation, transferred to another microcentrifuge tube, and stored at −20° C. Spleens are removed aseptically using sterile instruments and transferred to a petri dish containing 5 ml of RPMI medium.

Each excised spleen is dispersed into a single cell suspension and the cells are washed once in RPMI medium. The lymphocytes are counted and resuspended at a concentration of 5×10⁶ cells per ml. Cell cultures are set up in complete RMPI and incubated at 37° C. in 5% CO₂ under relatively high humidity. For cytokine analysis, cells are cultured in 48 well tissue culture cluster dishes (Costar, Cambridge, Mass.) at 5×10⁶ cells per well with 10 μg of the antigen of interest (heterologous target). The negative control wells contain cells alone in complete RPMI while the positive control wells contain cells stimulated with 1 μg per ml Concanavalin A. The supernatants are collected after 72 hours of incubation by centrifugation and stored at −70° C. until analyzed. Cytokine levels in the supernatants are measured as described above for DNA-immunized animals.

Quantitation of heterologous target-specific antibody responses. A standard ELISA is employed to measure antigen specific IgG1 and IgG2a antibody responses. Immulon 2.sup.HB microtiter plates (Corning, Park Ridge, Ill.) are coated with 0.2 μg per well of purified protein diluted in carbonate buffer (2.93 g sodium bicarbonate, 1.5 g sodium carbonate, 0.2 g sodium azide per liter; pH 9.5) by overnight incubation at 4° C. After extensive washing, the coated plates are blocked with 1% gelatin in PBS (8 g NaCl, 1.5 g Na₂ HPO₄, 0.2 g KH₂ PO₄, 0.2 g KCl per liter, pH 7.4) for one hour. After further extensive washing of the plates, the serum samples are added to appropriate wells at a dilution of 1:100 in PBS and incubated at 37° C. for 1.5 hours. After additional extensive washing, the plates are incubated with isotype-specific alkaline phosphatase-conjugated goat anti-mouse IgG1 or IgG2a (1:1000) (Southern Biotechnology Associates, Birmingham, Ala.) for 1 hour at 37° C. The plates are washed with PBS containing 0.5% Tween 20 (Sigma, St. Louis, Mo.), and then developed using alkaline phosphatase substrate (Sigma 140, Sigma). The optical density (OD) results are read at 405 nm and the date are presented as the average OD values of duplicate wells.

Statistical analysis. Statistical evaluations are performed by one-way analysis of variance or Mann Whitney test using InStat 2.0 (GraphPad Software, San Diego, Calif.), or other appropriate statistical tests.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. An immune response altering agent comprising:
(a) a first domain comprising one or more components selected from the group consisting of T cell epitopes, B cell epitopes, and TLR-binding proteins or TLR-binding domains thereof; and; and
(b) a second domain comprising a heterologous target molecule against which an immune response is desired;
wherein the first domain alters an immune response in a subject against the heterologous target.

2. The immune response altering agent of claim 1 wherein the T cell epitopes are derived from more than one source.

3. The immune response altering agent of claim 1 wherein the T cell epitopes are derived from one or more sources selected from the group consisting of an infectious agent, a virus, a bacterium, a tumor, an autoantigen, a fungus, a yeast, mycoplasma, a self antigen, and a non-self antigen.

4-13. (canceled)

14. The immune response altering agent of claim 1 wherein the first domain is covalently attached to the second domain via a peptide bond.

15. The immune response altering agent of claim 1 wherein the first domain is chemically coupled to the second domain.

16. The immune response altering agent of claim 1 wherein the first domain is noncovalently attached to the second domain.

17-20. (canceled)

21. The immune response altering agent of claim 1 wherein the first domain is attached to the second domain via biotin.

22. The immune response altering agent of claim 1 wherein the first domain is attached to the second domain via an antibody.

23. The immune response altering agent of claim 1 wherein the heterologous target comprises a protein.

24-27. (canceled)

28. The immune response altering agent of claim 1 wherein the heterologous target comprises a tumor antigen.

29. The immune response altering agent of claim 1 wherein the heterologous target comprises an autoantigen.

30-31. (canceled)

32. The immune response altering agent of claim 1 wherein the heterologous target comprises a S. pneumoniae protein.

33-37. (canceled)

38. The immune response altering agent of claim 1 wherein the T cell epitopes are generated synthetically.

39. The immune response altering agent of claim 1 wherein the T cell epitopes are generated recombinantly.

40. The immune response altering agent of claim 1 wherein the T cell epitopes comprise CD4⁺ T helper cell epitopes.

41. The immune response altering agent of claim 1 wherein the T cell epitopes comprise CD8⁺ cytotoxic T cell epitopes.

42. The immune response altering agent of claim 1 wherein the T cell epitopes comprise CD4⁺ T helper cell epitopes and CD8⁺ cytotoxic T cell epitopes.

43. The immune response altering agent of claim 1 wherein the agent is a polynucleotide encoding a fusion protein.

44. The immune response altering agent of claim 1 wherein the agent is a fusion protein.

45. A composition comprising an immune response altering agent of claim 1.

46. A composition comprising an immune response altering agent of claim 1 in combination with a physiologically acceptable excipient.

47. A composition comprising an immune response altering agent of claim 1 in combination with an adjuvant.

48-52. (canceled)

53. A method for inducing an immune response to a target comprising administering to a subject an immune response altering agent of claim 1.

54. The method of claim 53 wherein the immune response comprises a CD8 cytotoxic T cell mediated response.

55. The method of claim 53 wherein the immune response comprises a CD4 T helper cell mediated response.

56. The method of claim 53 wherein the immune response comprises predominantly a Th1 type response.

57. The method of claim 53 wherein the immune response comprises predominantly a Th2 type response.

58. A T cell epitope cassette comprising multiple T cell epitopes, wherein said cassette alters an immune response to a heterologous target when administered as a fusion with, or attached to the heterologous target.

59. A composition comprising:
(a) a heterologous target molecule; and
(b) one or more first domains, said one or more first domains comprising a polypeptide sequence selected from the group consisting of:
(i) either one of the full length polypeptide sequences set forth in SEQ ID NOs:214-215 (ESAT 6 and CFP10); and
(ii) a fragment of either one of the full length polypeptide sequences set forth in SEQ ID NO:214-215, wherein the fragment induces an immune response that is not substantially reduced as compared to an immune response induced by the full length polypeptide.

60-61. (canceled)

62. The composition of claim 59 wherein the polypeptide comprises the sequence set forth in SEQ ID NO:214 and the sequence set forth in SEQ ID NO:215.

63-64. (canceled)

65. The composition of claim 59 wherein the first domain comprises at least one polypeptide comprising any one or more of the sequences set forth in SEQ ID NOs:216-293.

66. A method for inducing or enhancing an immune response to a heterologous target molecule in an individual comprising administering to the individual a composition comprising:
(a) the heterologous target molecule; and
(b) one or more first domains, said one or more first domains comprising a polypeptide sequence selected from the group consisting of:
(i) either one of the full length polypeptide sequences set forth in SEQ ID NOs:214-215; and
(ii) a fragment of either one of the full length polypeptide sequences set forth in SEQ ID NO:214-215, wherein the fragment induces an immune response that is not substantially reduced as compared to an immune response induced by the full length polypeptide.

67. The method of claim 66 wherein the polypeptide comprises the sequence set forth in SEQ ID NO:215.

68. The method of claim 66 wherein the polypeptide comprises the sequence set forth in SEQ ID NO:216.

69. The method of claim 66 wherein the polypeptide comprises the sequence set forth in SEQ ID NO:215 and the sequence set forth in SEQ ID NO:216.

70-71. (canceled)

72. The method of claim 66 wherein the first domain comprises at least one polypeptide comprising any one or more of the sequences set forth in SEQ ID NOs:216-293.

73. The method of claim 66 wherein the immune response is a predominantly Th1-type response.

74. The method of claim 66 wherein the immune response is a predominantly Th2-type response.

75. The method of claim 66 wherein the immune response is a Th0-type response.

76. The method of claim 66 wherein the immune response is a CD4+ T cell response.

77. The method of claim 66 wherein the immune response is a CD8+ T cell response.

78. The method of claim 66 wherein the target molecule comprises an antigen selected from the group consisting of a viral coat protein, influenza neuraminidase, influenza hemmaglutinin, HIV gp160 or derivatives thereof, SARS coat protein, Herpes virion proteins, WNV capsid proteins, pneumococcal PsaA, PspA, LytA, Nisseria gonnorhea OMP or Nisseria gonnorhea surface proteases.

79. A method for inducing or enhancing an immune response to a target molecule in an individual comprising administering to the individual a composition comprising:
(a) the target molecule; and
(b) one or more polypeptides or fragments thereof, wherein said one or more polypeptides or fragments thereof comprise at least one T cell epitope.

80-81. (canceled)

82. The immune response altering agent of claim 1 wherein said agent is attached to a targeting molecule.

83. The immune response altering agent of claim 82 wherein said targeting molecule is an antibody or antigen-binding fragment thereof.


SEQUENCE LISTING (295 sequences)

SEQ ID NO: 1 - protein, 16 aa, Mycobacterium leprae
LEDPYEKIGAELVKEV

SEQ ID NO: 2 - protein, 15 aa, Mycobacterium leprae
EQIAATAAISAGDQS

SEQ ID NO: 3 - protein, 15 aa, Mycobacterium leprae
AGDQSIGDLIAEAMD

SEQ ID NO: 4 - protein, 15 aa, Mycobacterium leprae
VEGAGDTDAIAGRVA

SEQ ID NO: 5 - protein, 15 aa, Mycobacterium leprae
AGGVAVIKAGAATEV

SEQ ID NO: 6 - protein, 15 aa, Mycobacterium leprae
GDEATGANIVKVALE

SEQ ID NO: 7 - protein, 15 aa, Mycobacterium leprae
LQNAASIAGLFLTTE

SEQ ID NO: 8 - protein, 15 aa, Mycobacterium leprae
AGGGVTLLQAAPALD

SEQ ID NO: 9 - protein, 13 aa, Mycobacterium leprae
RVAQIRTEIENSD

SEQ ID NO: 10 - protein, 13 aa, Mycobacterium leprae
LLQAAPALDKLKL

SEQ ID NO: 11 - protein, 13 aa, Mycobacterium leprae
PEKTAAPASDPTG

SEQ ID NO: 12 - protein, 16 aa, Mycobacterium tuberculosis
LEDPYEKIGAELVKEV

SEQ ID NO: 13 - protein, 15 aa, Mycobacterium tuberculosis
EQIAATAAISAGDQS

SEQ ID NO: 14 - protein, 15 aa, Mycobacterium tuberculosis
AGDQSIGDLIAEAMD

SEQ ID NO: 15 - protein, 15 aa, Mycobacterium tuberculosis
VEGAGDTDAIAGRVA

SEQ ID NO: 16 - protein, 15 aa, Mycobacterium tuberculosis
AGGVAVIKAGAATEV

SEQ ID NO: 17 - protein, 15 aa, Mycobacterium tuberculosis
GDEATGANIVKVALE

SEQ ID NO: 18 - protein, 15 aa, Mycobacterium tuberculosis
LQNAASIAGLFLTTE

SEQ ID NO: 19 - protein, 15 aa, Mycobacterium tuberculosis
AGGGVTLLQAAPTLD

SEQ ID NO: 20 - protein, 13 aa, Mycobacterium tuberculosis
RVAQIRQEIENSD

SEQ ID NO: 21 - protein, 13 aa, Mycobacterium tuberculosis
LLQAAPTLDELKL

SEQ ID NO: 22 - protein, 13 aa, Mycobacterium tuberculosis
PEKTAEKASVPGG

SEQ ID NO: 23 - protein, 9 aa, Mycobacterium tuberculosis
LPAKFLEGF

SEQ ID NO: 24 - protein, 18 aa, Mycobacterium tuberculosis
YLQVPSPSMGRDIKVQFQ

SEQ ID NO: 25 - protein, 18 aa, Mycobacterium tuberculosis
GRDIKVQFQSGGNNSPAV

SEQ ID NO: 26 - protein, 18 aa, Mycobacterium tuberculosis
GCQTYKWETLLTSELPQW

SEQ ID NO: 27 - protein, 9 aa, Mycobacterium tuberculosis
IPAEFLENF

SEQ ID NO: 28 - protein, 9 aa, Mycobacterium tuberculosis
WPTLIGLAM

SEQ ID NO: 29 - protein, 9 aa, Mycobacterium tuberculosis
IPAKFLEGL

SEQ ID NO: 30 - protein, 9 aa, Mycobacterium tuberculosis
MPVGGQSSF

SEQ ID NO: 31 - protein, 10 aa, Mycobacterium tuberculosis
MPVGGQSSFY

SEQ ID NO: 32 - protein, 18 aa, Mycobacterium tuberculosis
MSQIMYNYPAMMAHAGDM

SEQ ID NO: 33 - protein, 16 aa, Mycobacterium tuberculosis
ITYQGWQTQWNQALED

SEQ ID NO: 34 - protein, 11 aa, Mycobacterium tuberculosis
ATFAAPVALAA

SEQ ID NO: 35 - protein, 11 aa, Mycobacterium tuberculosis
SGATIPQGEQS

SEQ ID NO: 36 - protein, 25 aa, Mycobacterium tuberculosis
AVAASNNPELTTLTAALSGQLNPQV

SEQ ID NO: 37 - protein, 21 aa, Mycobacterium tuberculosis
ALSGQLNPQVNLVDTLNSGQY

SEQ ID NO: 38 - protein, 26 aa, Mycobacterium tuberculosis
FSKLPASTIDELKTNSSLLTSILTYH

SEQ ID NO: 39 - protein, 24 aa, Mycobacterium tuberculosis
GNADVVCGGVSTANATVYMIDSVL

SEQ ID NO: 40 - protein, 15 aa, Mycobacterium tuberculosis
ATTVYMIDSVLMPPA

SEQ ID NO: 41 - protein, 20 aa, Mycobacterium tuberculosis
MTEQQWNFAGIEAAASAIQG

SEQ ID NO: 42 - protein, 13 aa, Mycobacterium tuberculosis
EQQWNFAGIEAAA

SEQ ID NO: 43 - protein, 9 aa, Mycobacterium tuberculosis
WNFAGIEAA

SEQ ID NO: 44 - protein, 20 aa, Mycobacterium tuberculosis
VQGVQQKWDATATELNNALQ

SEQ ID NO: 45 - protein, 24 aa, Mycobacterium tuberculosis
AWGGSGSEAYQGVQQKWDATATEL

SEQ ID NO: 46 - protein, 24 aa, Mycobacterium tuberculosis
QGVQQKWDATATELNNALQNLART

SEQ ID NO: 47 - protein, 24 aa, Mycobacterium tuberculosis
LARTISEAGQAMASTEGNVTGMFA

SEQ ID NO: 48 - protein, 13 aa, Mycobacterium tuberculosis
EQQWNFAGIEAAA

SEQ ID NO: 49 - protein, 20 aa, Streptococcus mutans
NNNDVNIDRTLVAKQSVVKF

SEQ ID NO: 50 - protein, 20 aa, Streptococcus mutans
QLKTADLPAGRDETTSFVLV

SEQ ID NO: 51 - protein, 20 aa, Streptococcus mutans
LATFNADLTKSVATIYPTVV

SEQ ID NO: 52 - protein, 8 aa, Chlamydia pneumoniae
GDYVFDRI

SEQ ID NO: 53 - protein, 9 aa, Chlamydia pneumoniae
SLLGNATAL

SEQ ID NO: 54 - protein, 9 aa, Chlamydia pneumoniae
QAVANGGAI

SEQ ID NO: 55 - protein, 9 aa, Chlamydia pneumoniae
RGAFCDKEF

SEQ ID NO: 56 - protein, 10 aa, Chlamydia pneumoniae
CYGRLYSVKV

SEQ ID NO: 57 - protein, 10 aa, Chlamydia pneumoniae
KYNEEARKKI

SEQ ID NO: 58 - protein, 9 aa, Chlamydia pneumoniae
GPKGRHVVI

SEQ ID NO: 59 - protein, 20 aa, Corynebacterium diptheriae
NLFQVVHWSYNRPAYSPGYV

SEQ ID NO: 60 - protein, 20 aa, Esherichia coli
AQTGNKTRLAFAGLKYADVG

SEQ ID NO: 61 - protein, 20 aa, Esherichia coli
FDFGLRPSIAYTKSKAKDVE

SEQ ID NO: 62 - protein, 21 aa, Esherichia coli
FEVGATYYFNKNMSTYVDYII

SEQ ID NO: 63 - protein, 20 aa, Esherichia coli
NKNMSTYVDYIINQIDSDNK

SEQ ID NO: 64 - protein, 9 aa, Eshericia coli
TPHPARIGL

SEQ ID NO: 65 - protein, 15 aa, Salmonella typhimurium
LIQCMLKKTMLSINQ

SEQ ID NO: 66 - protein, 9 aa, Listeria monocytogenes
GYKDGNEYI

SEQ ID NO: 67 - protein, 20 aa, Borrelia burgdorferi
VVKEGTVTLSKNISKSGEVS

SEQ ID NO: 68 - protein, 9 aa, Lymphocytic choriomeningitis virus
FQPQNGQFI

SEQ ID NO: 69 - protein, 9 aa, Lymphocytic choriomeningitis virus
RPQASGVYM

SEQ ID NO: 70 - protein, 9 aa, Lymphocytic choriomeningitis virus
PYIACRTSI

SEQ ID NO: 71 - protein, 10 aa, Lymphocytic choriomeningitis virus
MPYIACRTSI

SEQ ID NO: 72 - protein, 10 aa, Lymphocytic choriomeningitis virus
WPYIACRTSI

SEQ ID NO: 73 - protein, 13 aa, Lassa Fever Virus
FGTMPSLTLACLT

SEQ ID NO: 74 - protein, 13 aa, Lassa Fever Virus
FGTMPSLTIACMC

SEQ ID NO: 75 - protein, 13 aa, Lassa Fever Virus
QGQVDLNDAVQAL

SEQ ID NO: 76 - protein, 13 aa, Lassa Fever Virus
QGQADLNDVIQSL

SEQ ID NO: 77 - protein, 13 aa, Lassa Fever Virus
ALGMFISDTPGER

SEQ ID NO: 78 - protein, 13 aa, Lassa Fever Virus
SLGMFVSDTPGER

SEQ ID NO: 79 - protein, 13 aa, Lassa Fever Virus
QLDPNAKTWMDIE

SEQ ID NO: 80 - protein, 13 aa, Lassa Fever Virus
NLIPNAKTWMDIE

SEQ ID NO: 81 - protein, 13 aa, Lassa Fever Virus
VWDQYKDLCHMHT

SEQ ID NO: 82 - protein, 13 aa, Lassa Fever Virus
VWDQFKDLCHMHT

SEQ ID NO: 83 - protein, 13 aa, Lassa Fever Virus
IWDEYKHLCRMHT

SEQ ID NO: 84 - protein, 8 aa, HIV-1 Nef
FPVTPQVP

SEQ ID NO: 85 - protein, 9 aa, HIV-1 Nef
FPVTPRVPL

SEQ ID NO: 86 - protein, 9 aa, HIV-1 Nef
TPQVPLRPM

SEQ ID NO: 87 - protein, 9 aa, HIV-1 Nef
AVDLSHFLK

SEQ ID NO: 88 - protein, 9 aa, HIV-1 Nef
YPLTFGWCY

SEQ ID NO: 89 - protein, 9 aa, HIV-1 Nef
PLTFGWCYK

SEQ ID NO: 90 - protein, 9 aa, HIV-1 Nef
LTFGWCYKL

SEQ ID NO: 91 - protein, 9 aa, HIV-1 Gag
GEIYKRWII

SEQ ID NO: 92 - protein, 9 aa, HIV-1 Gag
EIYKRWIIL

SEQ ID NO: 93 - protein, 10 aa, HIV-1 Gag
KRWIILGLNK

SEQ ID NO: 94 - protein, 8 aa, HIV-1 Gag
ILGLNKIV

SEQ ID NO: 95 - protein, 11 aa, HIV-1 Gag
ILGLNKIVRMY

SEQ ID NO: 96 - protein, 18 aa, Hepatitis B virus
QAGFFLLTRILTIPQSLD

SEQ ID NO: 97 - protein, 20 aa, Hepatitis B virus
SCCCTKPTDGNCTCIPIPSS

SEQ ID NO: 98 - protein, 12 aa, Hepatitis B virus
WEWASVRFSWLS

SEQ ID NO: 99 - protein, 14 aa, Hepatitis B virus
LPLLPIFFCLWVYI

SEQ ID NO: 100 - protein, 9 aa, Human Papillomavirus E7
RAHYNIVTF

SEQ ID NO: 101 - protein, 35 aa, Human Papillomavirus E7
GQAEPDRAHYNIVTFCCKCDSTLRLCVQSTHVDIR

SEQ ID NO: 102 - protein, 9 aa, Epstein Barr virus
RRIYDLIEL

SEQ ID NO: 103 - protein, 9 aa, Epstein Barr virus
RKIYDLIEL

SEQ ID NO: 104 - protein, 9 aa, Epstein Barr virus
FRKAQIQGL

SEQ ID NO: 105 - protein, 8 aa, Epstein Barr virus
HRCQAIRK

SEQ ID NO: 106 - protein, 11 aa, Epstein Barr virus
RRARSLSAERY

SEQ ID NO: 107 - protein, 9 aa, Epstein Barr virus
RRRWRRLTV

SEQ ID NO: 108 - protein, 17 aa, Epstein Barr virus
DWTGGALLVLYSFALML

SEQ ID NO: 109 - protein, 10 aa, Epstein Barr virus
ALLVLYSFAL

SEQ ID NO: 110 - protein, 9 aa, Epstein Barr virus
LLVLYSFAL

SEQ ID NO: 111 - protein, 9 aa, Epstein Barr virus
ALLVLYSFA

SEQ ID NO: 112 - protein, 9 aa, Epstein Barr virus
VLYSFALML

SEQ ID NO: 113 - protein, 18 aa, Epstein Barr virus
LVLGIWIYLLEMLWRRLG

SEQ ID NO: 114 - protein, 9 aa, Epstein Barr virus
YLLEMLWRL

SEQ ID NO: 115 - protein, 17 aa, Epstein Barr virus
LIIALYLQQNWWTLLVD

SEQ ID NO: 116 - protein, 9 aa, Epstein Barr virus
IALYLQQNW

SEQ ID NO: 117 - protein, 9 aa, Epstein Barr virus
ALYLQQNWW

SEQ ID NO: 118 - protein, 9 aa, Epstein Barr virus
YLQQNWWTL

SEQ ID NO: 119 - protein, 9 aa, Epstein Barr virus
QNWWTLLVD

SEQ ID NO: 120 - protein, 9 aa, Epstein Barr virus
LYLQQNWWT

SEQ ID NO: 121 - protein, 17 aa, Epstein Barr virus
LIWMYYHGQRHSDEHHH

SEQ ID NO: 122 - protein, 9 aa, Epstein Barr virus
QRHSDEHHH

SEQ ID NO: 123 - protein, 9 aa, Epstein Barr virus
GQRHSDEHH

SEQ ID NO: 124 - protein, 9 aa, Epstein Barr virus
YYHGQRHSD

SEQ ID NO: 125 - protein, 9 aa, Epstein Barr virus
WMYYHGQRH

SEQ ID NO: 126 - protein, 17 aa, Epstein Barr virus
TDDSGHESDSNSNEGRH

SEQ ID NO: 127 - protein, 9 aa, Epstein Barr virus
ESDSNSNEG

SEQ ID NO: 128 - protein, 9 aa, Epstein Barr virus
DSNSNEGRH

SEQ ID NO: 129 - protein, 17 aa, Epstein Barr virus
PHSPSDSAGNDGGPPQL

SEQ ID NO: 130 - protein, 9 aa, Epstein Barr virus
AGNDGGPPQ

SEQ ID NO: 131 - protein, 9 aa, Epstein Barr virus
PSDSAGNDG

SEQ ID NO: 132 - protein, 17 aa, Epstein Barr virus
RHSDEHHHDDSLPHPQQ

SEQ ID NO: 133 - protein, 10 aa, Epstein Barr virus
EENLLDVFRM

SEQ ID NO: 134 - protein, 15 aa, Epstein Barr virus
LVSDYCNVLNKEFTA

SEQ ID NO: 135 - protein, 15 aa, Epstein Barr virus
FFIQAPSNRVMIPAT

SEQ ID NO: 136 - protein, 15 aa, Epstein Barr virus
RVMIPATIGTAMYKL

SEQ ID NO: 137 - protein, 15 aa, Epstein Barr virus
KHSRVRAYTYSKVLG

SEQ ID NO: 138 - protein, 15 aa, Epstein Barr virus
RALIKTLPRASYSSH

SEQ ID NO: 139 - protein, 15 aa, Epstein Barr virus
ERPIFPHPSKPTFLP

SEQ ID NO: 140 - protein, 15 aa, Epstein Barr virus
EVCQPKRIRPFHPPG

SEQ ID NO: 141 - protein, 15 aa, Epstein Barr virus
QKEEAAICGQMDLSH

SEQ ID NO: 142 - protein, 10 aa, Epstein Barr virus
DYCNVLNKEF

SEQ ID NO: 143 - protein, 9 aa, Epstein Barr virus
ATIGTAMYK

SEQ ID NO: 144 - protein, 9 aa, Epstein Barr virus
QAKWRLQTL

SEQ ID NO: 145 - protein, 9 aa, Epstein Barr virus
FLRGRAYGL

SEQ ID NO: 146 - protein, 9 aa, Epstein Barr virus
IVTDFSVIK

SEQ ID NO: 147 - protein, 10 aa, Epstein Barr virus
AVFDRKSDAK

SEQ ID NO: 148 - protein, 9 aa, Epstein Barr virus
RRIYDLIEL

SEQ ID NO: 149 - protein, 11 aa, Epstein Barr virus
RRARSLSAERY

SEQ ID NO: 150 - protein, 9 aa, Epstein Barr virus
LLWTLVVLL

SEQ ID NO: 151 - protein, 9 aa, Epstein Barr virus
CLGGLLTMV

SEQ ID NO: 152 - protein, 9 aa, Epstein Barr virus
IEDPPFNSL

SEQ ID NO: 153 - protein, 11 aa, Epstein Barr virus
SSCSSCPLSKI

SEQ ID NO: 154 - protein, 9 aa, Epstein Barr virus
TYGPVFMCL

SEQ ID NO: 155 - protein, 9 aa, Epstein Barr virus
APENAYQAY

SEQ ID NO: 156 - protein, 8 aa, Epstein Barr virus
RAKFKQLL

SEQ ID NO: 157 - protein, 9 aa, Epstein Barr virus
GLCTLVAML

SEQ ID NO: 158 - protein, 9 aa, Epstein Barr virus
TLDYKPLSV

SEQ ID NO: 159 - protein, 10 aa, Epstein Barr virus
QNGALAINTF

SEQ ID NO: 160 - protein, 10 aa, Epstein Barr virus
LLDFVRFMGV

SEQ ID NO: 161 - protein, 10 aa, Epstein Barr virus
EENLLDFVRF

SEQ ID NO: 162 - protein, 9 aa, Epstein Barr virus
HPLTNNLPL

SEQ ID NO: 163 - protein, 9 aa, Hantaan Virus
NAHEGQLVI

SEQ ID NO: 164 - protein, 9 aa, Hantaan Virus
ISNQEPLKL

SEQ ID NO: 165 - protein, 14 aa, Hepatitis C Virus
GYKVLVLNPSVAAT

SEQ ID NO: 166 - protein, 15 aa, Dengue Virus
LIGFRKEIGRMLNIL

SEQ ID NO: 167 - protein, 15 aa, Dengue Virus
KGPLRMVLAFITFLR

SEQ ID NO: 168 - protein, 15 aa, Rotavirus
RNFDTIRLSFQLVER

SEQ ID NO: 169 - protein, 14 aa, Rotavirus
RLSFQLVRPPNMTP

SEQ ID NO: 170 - protein, 14 aa, Rotavirus
VRPPNMTPAVANLF

SEQ ID NO: 171 - protein, 15 aa, Measles Virus
LSEIKGVIVHRLEGV

SEQ ID NO: 172 - protein, 11 aa, Measles Virus
INQDPDKILTY

SEQ ID NO: 173 - protein, 15 aa, Canine Distemper Virus
LSEVKGVIVHRLEAV

SEQ ID NO: 174 - protein, 11 aa, Canine Distemper Virus
INQSPDKILTY

SEQ ID NO: 175 - protein, 9 aa, Trypanosoma cruzi trans-sialidase
IYNVGQVSI

SEQ ID NO: 176 - protein, 15 aa, Trypanosoma cruzi
SHNFTLVASVIIEEA

SEQ ID NO: 177 - protein, 15 aa, Trypanosoma cruzi
LVASVIIEEAPSGNT

SEQ ID NO: 178 - protein, 20 aa, Toxoplasma gondii
TDPGDVVIEELFNRIPETSV

SEQ ID NO: 179 - protein, 18 aa, Toxoplasma gondii
LQLIRLAASLQHYGLVHA

SEQ ID NO: 180 - protein, 24 aa, Toxoplasma gondii
IEWIYRRCKNIPQPVRALLEGFLR

SEQ ID NO: 181 - protein, 17 aa, Babesia bovis
EYLVNKVLYMATMNYKT

SEQ ID NO: 182 - protein, 13 aa, Babesia bovis
EAPWYKRWIKKFR

SEQ ID NO: 183 - protein, 12 aa, Babesia bovis
FREAPQATKHFL

SEQ ID NO: 184 - protein, 15 aa, Babesia bovis
FREAPQATKHFLDEN

SEQ ID NO: 185 - protein, 15 aa, Babesia bovis
FREAPQATKHFLGEN

SEQ ID NO: 186 - protein, 29 aa, Babesia bovis
FVVSLLKKNVVRDPESNDVENFASQYFYM

SEQ ID NO: 187 - protein, 30 aa, Babesia bovis
VNSEKVDADDAGNAETQQLPDAENEVRADD

SEQ ID NO: 188 - protein, 20 aa, Plasmodium vivax
NFVGKFLELQIPGHTDLLHL

SEQ ID NO: 189 - protein, 20 aa, Plasmodium vivax
FNQLMHVINFHYDLLRANVH

SEQ ID NO: 190 - protein, 20 aa, Plasmodium vivax
LDMLKKVVLGLWKPLDNIKD

SEQ ID NO: 191 - protein, 20 aa, Plasmodium vivax
LEYYLREKAKMAGTLIIPES

SEQ ID NO: 192 - protein, 20 aa, Plasmodium vivax
KKIKAFLETSNNKAAAPAQS

SEQ ID NO: 193 - protein, 20 aa, Plasmodium vivax
SKDQIKKLTSLKNKLERRQN

SEQ ID NO: 194 - protein, 16 aa, Plasmodium falciparum
DPNANPNVDPNANPNV

SEQ ID NO: 195 - protein, 23 aa, Plasmodium falciparum
FGYRKPLDNIKDNVGKMEDYIKK

SEQ ID NO: 196 - protein, 23 aa, Plasmodium falciparum
SKLNSLNNPHNVLQNFSVFFNKK

SEQ ID NO: 197 - protein, 22 aa, Plasmodium falciparum
GYRKPLDNIKDNVGKMEDYIKK

SEQ ID NO: 198 - protein, 21 aa, Plasmodium falciparum
KLNSLNNPHNVLQNFSVFFNK

SEQ ID NO: 199 - protein, 21 aa, Plasmodium falciparum
TKILLKHYKGLVKYYNGESSP

SEQ ID NO: 200 - protein, 21 aa, Plasmodium falciparum
HGFKYLIDGYEEINELLYKLN

SEQ ID NO: 201 - protein, 20 aa, Plasmodium falciparum
VTHESYQELVKKLEALEDAV

SEQ ID NO: 202 - protein, 20 aa, Plasmodium falciparum
GLFHKEKMILNEEEITTKGA

SEQ ID NO: 203 - protein, 20 aa, Plasmodium falciparum
DSNIMNSINNVMDEIDFFEK

SEQ ID NO: 204 - protein, 23 aa, Plasmodium falciparum
DDYTEYKLTESIDNILVKMFKTN

SEQ ID NO: 205 - protein, 21 aa, Plasmodium falciparum
LTMSNVKNVSQTNFKSLLRNL

SEQ ID NO: 206 - protein, 19 aa, Plasmodium falciparum
HTLETVNISDVNDFQISKY

SEQ ID NO: 207 - protein, 19 aa, Plasmodium falciparum
DDEDLDEFKPIVQYDNFQD

SEQ ID NO: 208 - protein, 19 aa, Plasmodium falciparum
EENIGIKELEDLIEKNENL

SEQ ID NO: 209 - protein, 18 aa, Plasmodium falciparum
DDLDEGIEKSSEELSEEK

SEQ ID NO: 210 - protein, 15 aa, Plasmodium falciparum
IKKGKKYEKTKDNNF

SEQ ID NO: 211 - protein, 22 aa, Plasmodium falciparum
DNEILQIVDELSEDITKYFMKL

SEQ ID NO: 212 - protein, 35 aa, Plasmodium falciparum
EQQQSDLEQERLAKEKLQEQQSDLEQERRAKEKLQ

SEQ ID NO: 213 - protein, 8 aa, Chicken Ovalbumin
SIINFEKL

SEQ ID NO: 214 - protein, 95 aa, Mycobacteria tuberculosis
MTEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 215 - protein, 100 aa, Mycobacteria tuberculosis
MAEMKTDAATLAQEAGNFERISGDLKTQIDQVESTAGSLQGQWRGAAGTAAQAAVVRFQEAANKQKQELDEISTNIRQAGVQYSRADEEQQQALSSQMGF

SEQ ID NO: 216 - protein, 15 aa, Mycobacteria tuberculosis
MTEQQWNFAGIEAAA

SEQ ID NO: 217 - protein, 15 aa, Mycobacteria tuberculosis
QWNFAGIEAAASAIQ

SEQ ID NO: 218 - protein, 15 aa, Mycobacteria tuberculosis
AGIEAAASAIQGNVT

SEQ ID NO: 219 - protein, 15 aa, Mycobacteria tuberculosis
AAASAIQGNVTSIHS

SEQ ID NO: 220 - protein, 15 aa, Mycobacteria tuberculosis
AIQGNVTSIHSLLDE

SEQ ID NO: 221 - protein, 15 aa, Mycobacteria tuberculosis
NVTSIHSLLDEGKQS

SEQ ID NO: 222 - protein, 15 aa, Mycobacteria tuberculosis
IHSLLDEGKQSLTKL

SEQ ID NO: 223 - protein, 15 aa, Mycobacteria tuberculosis
LDEGKQSLTKLAAAW

SEQ ID NO: 224 - protein, 15 aa, Mycobacteria tuberculosis
KQSLTKLAAAWGGSG

SEQ ID NO: 225 - protein, 15 aa, Mycobacteria tuberculosis
TKLAAAWGGSGSEAY

SEQ ID NO: 226 - protein, 15 aa, Mycobacteria tuberculosis
AAWGGSGSEAYQGVQ

SEQ ID NO: 227 - protein, 15 aa, Mycobacteria tuberculosis
GSGSEAYQGVQQKWD

SEQ ID NO: 228 - protein, 15 aa, Mycobacteria tuberculosis
EAYQGVQQKWDATAT

SEQ ID NO: 229 - protein, 15 aa, Mycobacteria tuberculosis
GVQQKWDATATELNN

SEQ ID NO: 230 - protein, 15 aa, Mycobacteria tuberculosis
KWDATATELNNALQN

SEQ ID NO: 231 - protein, 15 aa, Mycobacteria tuberculosis
TATELNNALQNLART

SEQ ID NO: 232 - protein, 15 aa, Mycobacteria tuberculosis
LNNALQNLARTISEA

SEQ ID NO: 233 - protein, 15 aa, Mycobacteria tuberculosis
LQNLARTISEAGQAM

SEQ ID NO: 234 - protein, 15 aa, Mycobacteria tuberculosis
ARTISEAGQAMASTE

SEQ ID NO: 235 - protein, 15 aa, Mycobacteria tuberculosis
SEAGQAMASTEGNVT

SEQ ID NO: 236 - protein, 15 aa, Mycobacteria tuberculosis
QAMASTEGNVTGMFA

SEQ ID NO: 237 - protein, 20 aa, Mycobacteria tuberculosis
MTEQQWNFAGIEAAASAIQG

SEQ ID NO: 238 - protein, 20 aa, Mycobacteria tuberculosis
IEAAASAIQGNVTSIHSLLD

SEQ ID NO: 239 - protein, 20 aa, Mycobacteria tuberculosis
NVTSIHSLLDEGKQSLTKLA

SEQ ID NO: 240 - protein, 20 aa, Mycobacteria tuberculosis
EGKQSLTKLAAAWGGSGSEA

SEQ ID NO: 241 - protein, 20 aa, Mycobacteria tuberculosis
AAWGGSGSEAYQGVQQKWDA

SEQ ID NO: 242 - protein, 20 aa, Mycobacteria tuberculosis
YQGVQQKWDATATELNNALQ

SEQ ID NO: 243 - protein, 20 aa, Mycobacteria tuberculosis
TATELNNALQNLARTISEAG

SEQ ID NO: 244 - protein, 20 aa, Mycobacteria tuberculosis
NLARTISEAGQAMASTEGNV

SEQ ID NO: 245 - protein, 10 aa, Mycobacteria tuberculosis
MTEQQWNFAG

SEQ ID NO: 246 - protein, 10 aa, Mycobacteria tuberculosis
WNFAGIEAAA

SEQ ID NO: 247 - protein, 10 aa, Mycobacteria tuberculosis
IEAAASAIQG

SEQ ID NO: 248 - protein, 10 aa, Mycobacteria tuberculosis
SAIQGNVTSI

SEQ ID NO: 249 - protein, 10 aa, Mycobacteria tuberculosis
NVTSIHSLLD

SEQ ID NO: 250 - protein, 10 aa, Mycobacteria tuberculosis
HSLLDEGKQS

SEQ ID NO: 251 - protein, 10 aa, Mycobacteria tuberculosis
EGKQSLTKLA

SEQ ID NO: 252 - protein, 10 aa, Mycobacteria tuberculosis
LTKLAAAWGG

SEQ ID NO: 253 - protein, 10 aa, Mycobacteria tuberculosis
AAWGGSGSEA

SEQ ID NO: 254 - protein, 10 aa, Mycobacteria tuberculosis
SGSEAYQGVQ

SEQ ID NO: 255 - protein, 10 aa, Mycobacteria tuberculosis
YQGVQQKWDA

SEQ ID NO: 256 - protein, 10 aa, Mycobacteria tuberculosis
QKWDATATEL

SEQ ID NO: 257 - protein, 10 aa, Mycobacteria tuberculosis
TATELNNALQ

SEQ ID NO: 258 - protein, 10 aa, Mycobacteria tuberculosis
NNALQNLART

SEQ ID NO: 259 - protein, 10 aa, Mycobacteria tuberculosis
NLARTISEAG

SEQ ID NO: 260 - protein, 10 aa, Mycobacteria tuberculosis
ISEAGQAMAS

SEQ ID NO: 261 - protein, 10 aa, Mycobacteria tuberculosis
QAMASTEGNV

SEQ ID NO: 262 - protein, 10 aa, Mycobacteria tuberculosis
TEGNVTGMFA

SEQ ID NO: 263 - protein, 20 aa, Mycobacteria tuberculosis
MAEMKTDAATLAQEAGNFER

SEQ ID NO: 264 - protein, 20 aa, Mycobacteria tuberculosis
LAQEAGNFERISGDLKTQID

SEQ ID NO: 265 - protein, 20 aa, Mycobacteria tuberculosis
ISGDLKTQIDQVESTAGSLQ

SEQ ID NO: 266 - protein, 20 aa, Mycobacteria tuberculosis
QVESTAGSLQGQWRGAAGTA

SEQ ID NO: 267 - protein, 20 aa, Mycobacteria tuberculosis
GQWRGAAGTAAQAAVVRFQE

SEQ ID NO: 268 - protein, 20 aa, Mycobacteria tuberculosis
AQAAVVRFQEAANKQKQELD

SEQ ID NO: 269 - protein, 20 aa, Mycobacteria tuberculosis
AANKQKQELDEISTNIRQAG

SEQ ID NO: 270 - protein, 20 aa, Mycobacteria tuberculosis
EISTNIRQAGVQYSRADEEQ

SEQ ID NO: 271 - protein, 20 aa, Mycobacteria tuberculosis
VQYSRADEEQQQALSSQMGF

SEQ ID NO: 272 - protein, 15 aa, Mycobacteria tuberculosis
MAEMKTDAATLAQEA

SEQ ID NO: 273 - protein, 15 aa, Mycobacteria tuberculosis
KTDAATLAQEAGNFE

SEQ ID NO: 274 - protein, 15 aa, Mycobacteria tuberculosis
ATLAQEAGNFERISG

SEQ ID NO: 275 - protein, 15 aa, Mycobacteria tuberculosis
QEAGNFERISGDLKT

SEQ ID NO: 276 - protein, 15 aa, Mycobacteria tuberculosis
NFERISGDLKTQIDQ

SEQ ID NO: 277 - protein, 15 aa, Mycobacteria tuberculosis
ISGDLKTQIDQVEST

SEQ ID NO: 278 - protein, 15 aa, Mycobacteria tuberculosis
LKTQIDQVESTAGSL

SEQ ID NO: 279 - protein, 15 aa, Mycobacteria tuberculosis
IDQVESTAGSLQGQW

SEQ ID NO: 280 - protein, 15 aa, Mycobacteria tuberculosis
ESTAGSLQGQWRGAA

SEQ ID NO: 281 - protein, 15 aa, Mycobacteria tuberculosis
GSLQGQWRGAAGTAA

SEQ ID NO: 282 - protein, 15 aa, Mycobacteria tuberculosis
GQWRGAAGTAAQAAV

SEQ ID NO: 283 - protein, 15 aa, Mycobacteria tuberculosis
GAAGTAAQAAVVRFQ

SEQ ID NO: 284 - protein, 15 aa, Mycobacteria tuberculosis
TAAQAAVVRFQEAAN

SEQ ID NO: 285 - protein, 15 aa, Mycobacteria tuberculosis
AAVVRFQEAANKQKQ

SEQ ID NO: 286 - protein, 15 aa, Mycobacteria tuberculosis
RFQEAANKQKQELDE

SEQ ID NO: 287 - protein, 15 aa, Mycobacteria tuberculosis
AANKQKQELDEISTN

SEQ ID NO: 288 - protein, 15 aa, Mycobacteria tuberculosis
QKQELDEISTNIRQA

SEQ ID NO: 289 - protein, 15 aa, Mycobacteria tuberculosis
LDEISTNIRQAGVQY

SEQ ID NO: 290 - protein, 15 aa, Mycobacteria tuberculosis
STNIRQAGVQYSRAD

SEQ ID NO: 291 - protein, 15 aa, Mycobacteria tuberculosis
RQAGVQYSRADEEQQ

SEQ ID NO: 292 - protein, 15 aa, Mycobacteria tuberculosis
VQYSRADEEQQQALS

SEQ ID NO: 293 - protein, 16 aa, Mycobacteria tuberculosis
RADEEQQQALSSQMGF

SEQ ID NO: 294 - protein, 13 aa, Salmonella typhimurium
LQRVRELAVQSAN

SEQ ID NO: 295 - protein, 17 aa, Salmonella typhimurium
LQKIDAALAQVDTLRSD